(19) United States
(12) Patent Application Publication
(10) Pub. No.: US 2023/0397928 A1
(43) Pub. Date: Dec. 14, 2023

(54) Skin Core Micro-Coring System

(71) Applicant: Sperry Bio, Inc., Springfield, VT (US)

(72) Inventors: Anders H. Carlsson, San Antonio, TX (US); Rodney K. Chan, San Antonio, TX (US); Charles R. Sperry, Chester, CT (US); Ayn Lavagnino, Camas, WA (US); Karl Wassmann, Dover, MA (US); Vincent A. Piucci, Oakham, MA (US); Mark Torselli, Keene, NH (US); Darren Berrouard, Washington, NH (US)

(73) Assignee: Sperry Bio, Inc., Springfield, VT (US)

(21) Appl. No.: 18/204,608

(22) Filed: Jun. 1, 2023

Related U.S. Application Data
(60) Provisional application No. 63/348,829, filed on Jun. 3, 2022.

(51) Int. Cl. A61B 17/322

(52) U.S. Cl. CPC A61B 17/322

(57) ABSTRACT

Arrangements of the innovation relate to a micro-coring system configured to harvest a plurality of cores, either sequentially or simultaneously. In one arrangement, the micro-coring system comprises a harvesting tool configured to harvest and transfer at least one tissue core from a donor site to a recipient site, the at least one tissue core having a diameter of between about 1.0 mm and 3.0 mm diameter. In one arrangement, the harvesting tool comprises: a coring tool configured to penetrate tissue at the donor site and capture a tissue core from the tissue, and an extractor pin disposed within an aperture extending along a longitudinal axis of the coring tool, the extractor pin configured to extract the core from the coring tool.

This patent application claims the benefit of U.S. Provisional Application No. 63/348,829, filed on Jun. 3, 2023, entitled “Skin Core Micro-Coring System,” the contents and teachings of which are hereby incorporated by reference in their entirety.

### BACKGROUND

Skin grafting is a surgical procedure that involves removing skin or other tissue from one area of a patient's body (e.g., donor site) and transplanting it to an area of the body (e.g., recipient site or wound site) where the tissue has been damaged due to burns, injury, infection, illness, birth defects, surgical procedures or other causes. In certain cases, the donor site and recipient site are located on the same patient's body, referred to as an autograft.

One type of skin graft, known as a split-thickness skin graft (STSG), includes a section of the epidermis, as well as a portion of the dermis. These layers are typically harvested from an area of healthy skin at a donor site, such as the front or outer thigh, abdomen, buttocks, or back of the donor. Split-thickness grafts are typically utilized to cover relatively large areas of a recipient site while minimizing damage to the donor site. The donor site heals with scarring by re-epithelialization from the dermis and surrounding skin and requires dressings during the healing process.

One type of skin graft, known as a full thickness graft (FTSG) includes a section of the epidermis and all of the dermis from the donor site. These layers are typically harvested from an area of healthy skin at a donor site, such as the abdomen, groin, forearm, or area above the clavicle. Full thickness grafts are generally used for small wounds on highly visible parts of the body, such as the face and hands. Unlike split thickness grafts, at the recipient site these grafts blend in well with the skin around them, resulting in regenerative healing with near normal skin function (including sensation, contractility, lubrication and heat loss) and cosmetic appearance at both the donor and recipient sites.

### SUMMARY

There are four basic tissue types. These include epithelial, connective, nervous, and muscle tissue. “Skin” is an exemplary term used in this disclosure for clarity, but the term is applicable to all tissue types. The terms “skin” and “tissue” both refer to any of these tissue types.

Conventional skin grafts can suffer from a variety of deficiencies. For example, a split thickness graft typically contains only a partial section of dermis, and therefore a thin epithelial layer, which lacks certain elements of normal skin. As such, the skin at the recipient site generally lacks the structure and appearance of normal skin and may lack sensory feeling and the ability to sweat and grow hair. In another example, full thickness grafts are typically relatively small in area because there are few areas of the body in which full thickness skin can be harvested and the resulting wound at the donor site can heal by primary closure, resulting in a small, clean defect. As such, the donor site is generally sutured closed directly.

In order to overcome the size limitation of full thickness grafts, certain conventional technologies for the harvesting of multiple, small diameter, full thickness columns of skin from the donor site. During operation of such technologies, relatively small diameter cores are removed at a donor site and these cores are typically separated by larger spaces of healthy skin. This results in minimal to no bleeding at the donor site, and allows rapid and complete healing of the skin tissue. At the recipient site, these full thickness cores promote healthy and substantially normal skin. However, these emerging technologies can also suffer from a variety of deficiencies. For example, skin cores are typically harvested individually from the donor site using a biopsy punch. Because a skin graft may require dozens, or even several hundred cores, the process can be extremely time consuming. Further, the cores are fragile and must be handled with great care. The relatively large quantity of individual cores that are extracted can be very difficult to control and to orient correctly, whether lying flat or in an anatomical epidermal-dermal orientation, and then maintain them in that orientation during transfer to the recipient site.

By contrast to conventional skin harvesting and grafting technologies, arrangements of the present innovation relate to a micro-coring system that can be configured to harvest a plurality of cores, either sequentially or simultaneously. In one arrangement the micro-coring system can harvest full thickness skin graft cores from a donor site and transfer these cores directly onto a carrier medium in a predetermined pattern and at a predetermined density. Following the transfer, the resulting micro-core autograft can be preserved as viable skin and efficiently transferred to a recipient site. In some arrangements, the micro-coring system can transfer the cores directly onto the wound site.

In one arrangement, a surgical system includes a guidance grid or support, a carrier medium and a micro-coring system. The support defines a grid of openings disposed over a donor site. A transfer medium is located between sections of the honeycomb-like support during harvesting. During the harvesting operation, the micro-coring system passes a harvesting tool along a single axis through the carrier medium and into the skin at the donor site to a predetermined depth where it captures and removes a core from the donor site. The harvesting tool retracts until the top of the core is disposed at the top of the carrier medium. The harvesting tool then releases the core so that it is captured by the medium. This process continues through each opening in the support until the carrier medium is fully populated by cores. Once complete, the populated medium, termed a micro-core autograft, is removed from the micro-coring system and placed onto the wound at the recipient site. Various arrangements may utilize a single harvesting tool, or multiple harvesting tools that work in unison or sequentially.

In one arrangement, the functions of the micro-coring system are controlled by a computerized device having a microcomputer-based controller (e.g., a memory and a processor). The X, Y and Z motions of the harvesting tool can be accomplished using, for example, stepper motors. The harvesting tool can include a coring tool and an ejector pin. The coring tool is configured to pierce the skin and capture the core. The coring tool may have additional motions to aid in penetrating the skin, such as an oscillating or rotational motion or spinning action that adds to the shearing capability of the coring tool. In some embodiments, an ultrasonic vibration of the coring tool may be used to aid penetration. The ejector pin holds the core in place during removal from the coring tool. In this arrangement, the harvesting tool can index in both the X and Y directions in order to travel to each opening in the grid. Additional attributes disclosed include methods to hold the core in the coring tool during extraction and a vacuum system to secure the device on the patient's skin during harvesting.

The carrier medium can be manufactured from a material that causes the micro-cores to stay in place on the medium and is used to hold and transfer the cores from the harvesting device to the recipient site. This medium acts as a wound dressing, holding the transferred cores in place, maintaining the pattern and density of cores with which they were transferred. In this manner, the micro-cores remain in position during transfer to the recipient site and until re-epithelialization takes place (generally two to three weeks), maintaining the same pattern of cores as when they were transferred from the harvester. This wound dressing mitigates the micro-cores from moving during this process, as any movement of the micro-cores may leave an area to heal by secondary intention, causing a scar to form. Secondary intention healing means a section of the wound is left open and must heal by itself, filling in and closing up naturally. Placing the cores into or onto a carrier medium provides two advantages. The medium supports the cores and provides a vehicle to transfer these cores to a recipient site, retaining both vertical and axial orientation. Once placed, the carrier medium protects the wound to allow for natural healing.

The carrier medium may be a commercially available product. This can be a skin regeneration product, for example, Integra® Dermal Regeneration Template, manufactured by Integra LifeSciences, or a wound dressing, for example, Opsite IV 3000 Dressing manufactured by Smith & Nephew or Telfa Clear Non-Adherent Clear Wound Dressing manufactured by Covidien.

In some arrangements, a manufactured scaffolding may be used as a carrier medium. This can be manufactured from polymers or other suitable materials using 3D printing or other suitable methods. In these arrangements, the scaffolding may be removed once the cores have been placed onto the recipient site, or may remain in place while the tissue heals. In some arrangements, the carrier medium and/or scaffolding may be made of a bioabsorbable material. This is a biocompatible material that is absorbed into the body over a period of time, a few weeks for example, and does not require removal. Other arrangements of the current innovation can utilize guidance grids of any desirable size, aspect and number of openings, as well as seeding density.

Some arrangements do not utilize a guidance grid. In these arrangements, the cores are transferred onto the carrier medium and are orientated by the harvesting device itself. In some arrangements, no medium is used, and the cores are transferred directly onto the recipient site by the harvesting device.

Some arrangements of the current innovation do not use computer guidance, and are operated manually, or can be powered by a motor or the like.

Arrangements of the innovation relate to a micro-coring system configured to harvest a plurality of cores, either sequentially or simultaneously. In one arrangement, the micro-coring system comprises a harvesting tool configured to harvest and transfer at least one tissue core from a donor site to a recipient site, the at least one tissue core having a diameter of between about 1.0 mm and 3.0 mm diameter. In one arrangement, the harvesting tool comprises: a coring tool configured to penetrate tissue at the donor site and capture a tissue core from the tissue, and an extractor pin disposed within an aperture extending along a longitudinal axis of the coring tool, the extractor pin configured to extract the core from the coring tool.

Arrangements of the innovation relate to a method of harvesting a tissue cores from a donor site, comprising: disposing a harvesting tool at a donor site, the harvesting tool comprising a coring tool defining an aperture extending along a longitudinal axis of the coring tool and an extractor pin disposed within the aperture defined by the coring tool; translating the coring tool in a first direction relative to the extractor pin and the donor site to penetrate tissue of the donor site; excising a tissue core from the tissue of the donor site, the tissue core having a diameter of between about 1.0 mm and 3.0 mm diameter; and translating the coring tool in a second direction relative to the extractor pin and the donor site to dispose the tissue core within the harvesting tool such that a top surface of the tissue core is disposed in proximity to the extractor pin.

Throughout this disclosure, the term “core” refers to a column of tissue that can be round, square, or other shaped. The terms “carrier medium” and “medium” may be used interchangeably. The terms “recipient site” and “wound site” may be used interchangeably.

### BRIEF DESCRIPTION OF THE DRAWINGS

The foregoing and other objects, features and advantages will be apparent from the following description of particular arrangements of the innovation, as illustrated in the accompanying drawings. The drawings are not necessarily to scale, emphasis instead being placed upon illustrating the principles of various arrangements of the innovation.

FIG. 1 illustrates a schematic representation of an arrangement of a micro-coring system.

FIG. 2 illustrates a side view of an arrangement of a harvesting tool.

FIG. 3 illustrates a cross sectional view of an arrangement of a harvesting tool through section 1-1 of FIG. 2.

FIG. 4 illustrates a top view of an arrangement of a grid section of a guidance grid.

FIG. 5 illustrates a cross sectional side view of an arrangement of the grid section taken through section 2-2 of FIG. 4.

FIG. 6 illustrates an enlarged cross sectional side view of an arrangement of the grid section taken from detail A of FIG. 5.

FIG. 7 illustrates an isometric view of a section of an arrangement of the grid section of FIG. 4.

FIG. 8 illustrates an isometric view of an arrangement of a guidance grid in the open position.

FIG. 9 illustrates an isometric view of an arrangement of a guidance grid in the closed position.

FIG. 10 illustrates an isometric view of an arrangement of a vacuum frame.

FIG. 11 illustrates a side view of an arrangement of a vacuum frame.

FIG. 12 illustrates an isometric view of an arrangement of a guidance grid with a vacuum frame installed.

FIGS. 13a through 13e illustrate a cross sectional side view of an arrangement of the steps of harvesting a core.

FIG. 14 illustrates a cross sectional side view of an arrangement of a carrier medium, core and skin tissue.

FIG. 15 illustrates an isometric view of an arrangement of a populated carrier medium.

FIGS. 16a through 16d illustrate a cross sectional side view of an example process for harvesting a core.

FIG. 17 illustrates an isometric view of an arrangement of a harvesting assembly.

FIG. 18 illustrates a top view of an arrangement of the harvesting assembly of FIG. 17.

FIG. 19 illustrates a cross sectional side view of an arrangement of the harvesting tool of FIG. 18 through section 3-3.

FIG. 20a illustrates an isometric view of an arrangement of a harvesting tool.

FIG. 20b illustrates a side view of an arrangement of the harvesting tool of FIG. 20a.

FIG. 20c illustrates a section view of the harvesting tool of FIG. 20b through section 4-4.

FIG. 20d illustrates a sectional view of the harvesting tool of FIG. 20b according to one arrangement.

FIG. 21 illustrates an isometric view of an arrangement of a micro-coring device.

FIG. 22 illustrates an isometric view of an arrangement of the micro-coring device of FIG. 21 in a retracted position.

FIG. 23 illustrates an isometric view of an arrangement of the micro-coring device of FIG. 21 in an extended position with a top of the housing removed.

FIG. 24 illustrates an isometric bottom view of an arrangement of the micro-coring device of FIG. 21 in a retracted position.

FIG. 25 illustrates an isometric bottom view of an arrangement of the micro-coring device of FIG. 21 in an extended position.

FIG. 26a illustrates a top isometric view of an arrangement of a micro-coring device having multiple automated harvesting tools.

FIG. 26b illustrates a bottom isometric view of an arrangement of the micro-coring device of FIG. 26a.

FIG. 27 illustrates a top isometric view of an arrangement of the micro-coring device of FIG. 26a with the housing top cover removed.

FIG. 28 illustrates a bottom view of an arrangement of the micro-coring device of FIG. 26a with the bottom cover removed.

FIG. 29a illustrates an example of the shape of the damaged tissue at a wound site.

FIG. 29b illustrates a carrier medium material.

FIG. 29c illustrates a portion of the carrier medium material of FIG. 29b.

FIG. 30 illustrates a plan view of an arrangement of the carrier medium material of FIG. 29c as placed onto a core manager tray.

FIG. 31 illustrates an arrangement of the core manager tray and carrier medium material of FIG. 30 disposed in a core manager in a first indexed position.

FIG. 32 illustrates an arrangement of the core manager tray and carrier medium material of FIG. 30 disposed in the core manager in a second indexed position.

FIG. 33 illustrates an arrangement of the core manager tray and carrier medium material of FIG. 30 disposed in the core manager in a third indexed position.

FIG. 34 illustrates an arrangement of the core manager with the core manager tray and carrier medium material of FIG. 30 removed.

FIG. 35a illustrates an isometric view of an arrangement of a coring tool.

FIG. 35b illustrates an isometric view of an arrangement of a coring tool.

FIG. 35c illustrates an isometric view of an arrangement of a coring tool.

FIG. 36 illustrates a bottom isometric view of an arrangement of a manual micro-coring device.

FIG. 37 illustrates a top view of the arrangement of a manual micro-coring device of FIG. 36.

FIG. 38 illustrates an isometric view of an arrangement of the micro-coring device of FIG. 36 with the top housing removed.

FIG. 39 illustrates a top view of the micro-coring device of FIG. 38.

FIG. 40 illustrates an end view of an arrangement of a device base with extractor pins.

FIG. 41a illustrates a side view of an arrangement of a harvesting tool.

FIG. 41b illustrates a section view taken through section 5-5 of FIG. 41a of an arrangement of a harvesting tool.

FIG. 42 illustrates an isometric view of an arrangement of a micro-coring device.

FIG. 43 illustrates an isometric view of the coring device of FIG. 42 with the top housing removed.

FIG. 44 illustrates an enlarged section view through one row of an arrangement of coring tools.

FIG. 45 illustrates an isometric view of an arrangement of an extraction device.

FIG. 46 illustrates an isometric view of an arrangement of an extraction device aligned with a micro-coring device.

FIG. 47 illustrates a side view of an arrangement of a micro-coring device with an extraction device inserted.

FIG. 48 illustrates a top view of an arrangement of an autograft.

FIG. 49 illustrates a top view of an arrangement of an autograft.

### DETAILED DESCRIPTION

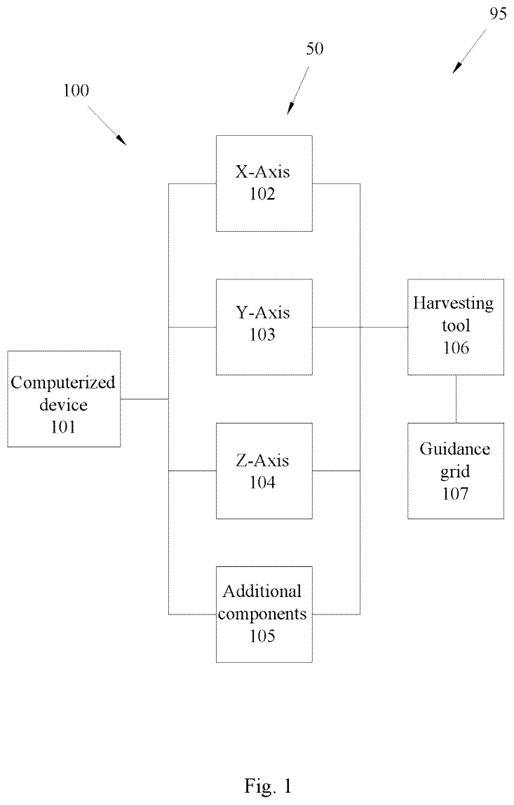

FIG. 1 illustrates a schematic representation of the components of a surgical system 95, according to one arrangement. For example, the surgical system 95 can include a skin core micro-coring system 100 and a guidance grid or support 107.

The skin core micro-coring system 100 can include a computerized device 101 having a controller (e.g., a processor and a memory), one or more power supplies, and other components used during operation of the micro-coring system 100. In one arrangement, the computerized device 101 can include a circuit board containing a 64-bit ARM microprocessor with 1.5 gigahertz clock speed, 1 Giga byte of read only memory, 40-Pin general purpose input/output, dedicated pulse width modulation (PWM) driver chip to control stepper motors via a 12C interface bus, 12V DC battery, HDMI port to support display, Bluetooth connectivity, and USB port for loading factory firmware revisions. Firmware to operate the computerized device 101 may be programed in a high-level language such as Python. Features can include, for example, internal software tests and checks, error and interrupt handling, fault detection, tolerance, and recovery characteristics, safety requirements and timing and memory requirements. The firmware can signal a ready/not ready LED light indicator or notice on a display for the operator to know whether the system has run diagnostics and is ready to go. In one arrangement a smart phone application can enable all control and information through that smart phone. Features may include the ability to enter and change parameters, reporting of operational progress, data collection and analysis, communication to a database, etc.

The micro-coring system 100 can also include actuators 50 disposed in electrical communication with the controller 101 and configured to adjust the velocity of movement and positioning of a harvesting tool 106 in three dimensions. For example, an X-axis actuator 102 is configured to adjust side to side movement of the harvesting tool 106, a Y-axis actuator 103 is configured to adjust fore and aft movement of the harvesting tool 106 (e.g., along a direction that is perpendicular to that generated by the X-axis actuator 102), and a Z-axis actuator 104 is configured to adjust vertical movement of the harvesting tool 106 relative to the guidance grid or support 107. In one arrangement, these actuators 102, 103, 104 are configured as stepper motors, for example direct current motors, which can move the harvesting tool 106 in small, discrete steps to achieve precise positioning relative to the guidance grid 107, as well as speed control. Positioning sensors such as potentiometers, optical sensors or other devices can be used to provide a feedback signal, which relates to the current X-Y-Z location of the harvesting tool 106, back to the computerized device 101.

The micro-coring system 100 can also include additional components 105 disposed in electrical communication with the computerized device 101. These components 105 may include, but are not limited to displays, LEDs or other visual aids, audible aids, communication mechanisms and oscillating or rotational movements, for example. The micro-coring system 100 can also be configured with an internal structure that guides the three-dimensional movement of the harvesting tool 106 and any other motions as necessary. In one arrangement, the micro-coring system 100 is enclosed in a medical grade plastic housing with dimensions of approximately 125 mm×110 mm×80 mm high.

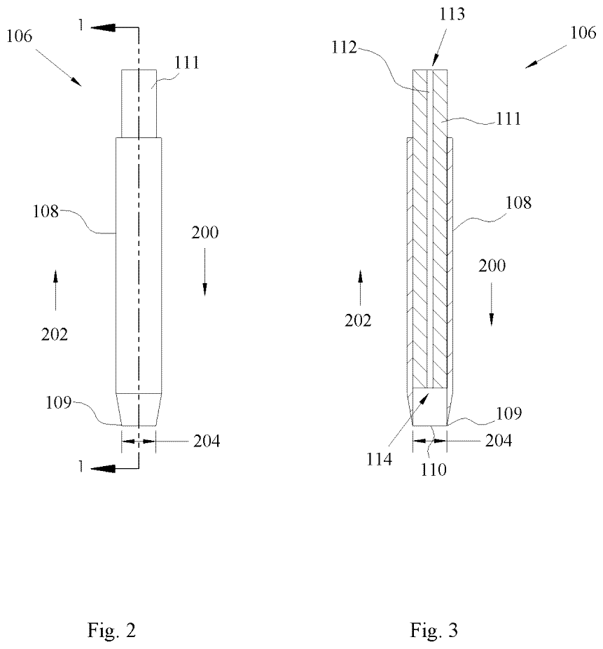

In one arrangement, the harvesting tool 106 is configured to pass through a carrier medium and into the skin at the donor site, where it harvests a core, then transfers that core to the carrier medium. As illustrated in the example provided by FIGS. 2 and 3, the harvesting tool 106 can include a coring tool 108 and an extractor pin 111, as described below. While the harvesting tool 106 is shown as a cylinder, tools of other shapes, for example, square, oval, elliptical, star-shaped, etc. are anticipated and remain within the scope of this innovation.

In this arrangement, the coring tool 108 is configured as a hollowed, generally cylindrical shaft having a sharpened cutting edge 109 disposed at a distal end. During operation, the computerized device 101 provides a signal to the Z-axis actuator 104 which, in turn, drives the coring tool 108 along direction 200 into the skin at the donor site. As the cutting edge 109 pierces the skin, a column of skin tissue (e.g., core) enters through an opening 110 defined by the coring tool 108 and fills the coring tool 108 to the depth of the insertion. To aid the coring tool 108 in penetrating the skin, the Z-axis actuator 104 can provide additional motion to the coring tool such as, for example, a reciprocating rotational motion (e.g., oscillation). This oscillating motion creates a shearing effect on the cutting edge 109 that aids in cutting through skin tissue. Other motions, such as spinning (e.g., fill rotation), ultrasonic vibration, etc. can be used. During operation, as the Z-axis actuator 104 retracts the coring tool 108 along direction 202, the core remains within the coring tool 108 and is extracted from the donor site.

In one arrangement, the extractor pin 111 is slidably disposed within the coring tool 108 and is configured as a stripper to position the harvested core within the carrier medium. The vertical motions of the coring tool 108 and extractor pin 111 can be actuated independently by the Z-axis actuator 104. As a result, with the retraction of the extractor pin 111 disposed in a first position relative to the coring tool 108 as the harvesting tool 106 advances into the skin, the core can enter the opening 110 of the coring tool 108. When the harvesting tool 106 is retracted along direction 202 to position the core at the desired position within the carrier medium, the extractor pin 111 is configured to translate at the same rate as the cutting tool 108 to hold the core in position. Once the core is disposed at the desired location in the carrier medium, the harvesting tool 106 can continue to translate along direction 202 while the extractor pin 111 remains stationary relative to the cutting tool 108 to dispose the core in position within the carrier medium. In another arrangement, the extractor pin 111 is configured to remain stationary at the desired core placement at the top of the carrier medium and only the coring tool 108 translates. In this arrangement, the coring tool 108 penetrates the tissue and captures a core. The coring tool 108 then retracts. Once the core makes contact with the extractor pin 111, it is held in that position by the extractor pin 111 as the coring tool 108 fully retracts, leaving the core in place in the carrier medium. In some arrangements, the core, rather than being placed within the carrier medium, may be placed on top of the carrier medium, directly onto the wound site, etc.

In one arrangement, the extractor pin 111 defines an aperture 112 that extends along its length. The aperture 112 is configured to allow air to escape as the core enters the coring tool 108. In one arrangement, when the harvesting tool 106 has reached the desired depth, a vacuum can be applied at the proximal end 113 of the extractor pin 111. This creates a suction that can selectively hold the core in place against the distal end 114 of the extractor pin 111 for harvesting or can release the core for transfer. Other mechanisms for holding and releasing cores, such as hydraulic or mechanical, can be used by the harvesting tool 106 and are within the scope of this innovation.

In one arrangement, the coring tool 108 has an inner diameter of between about 1.0 mm and 3.0 mm. The length can be defined by the specific application and design of the coring tool 108. The extractor pin 111 has an outer diameter configured to create a slip fit within the coring tool 108. This design can extract a skin core having a core diameter of between about 1.0 mm and 3.0 mm from a donor site. Core diameters may be chosen for specific applications.

In some arrangements, during operation, the coring tool 108 is configured to pierce the carrier medium prior to being advanced by the harvesting tool 106 through an opening in the guidance grid 107. In some arrangements, a top layer of the carrier medium is formed of silicone. When the coring tool 108 penetrates the carrier medium as it harvests a core, the coring tool 108 can extract a piece of the carrier medium material from the carrier medium prior to extracting each skin core from the donor site. As such, following extraction from the donor site, each core disposed within the carrier medium can include a piece of silicone material on its top. In one arrangement, the micro-coring system 100 is configured to limit or prevent silicone material elements from being extracted from the carrier medium during insertion of the coring tool 108.

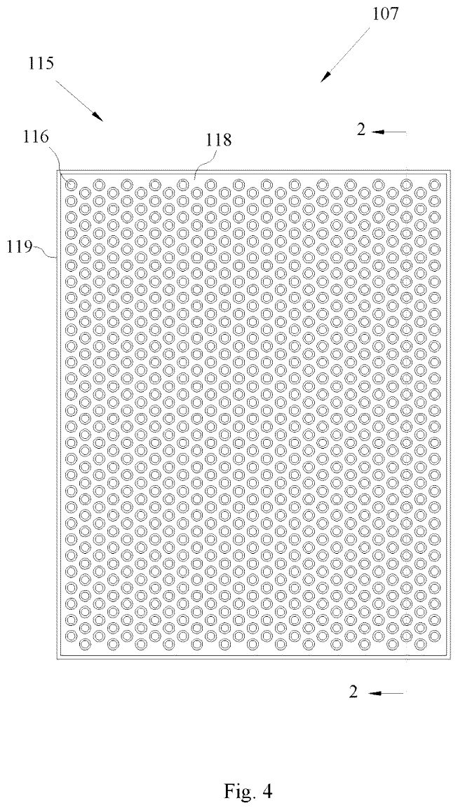

For example, a carrier medium preparation tool (not shown), such as a knife blade, can be mounted on a structure attached to the harvesting tool 106 in a manner that locates the carrier medium preparation tool above an opening 116 (FIG. 4) defined by the guidance grid 107 that is subsequent to the opening 116 through which the harvesting tool 106 is entering. The micro-coring system 100 can actuate the carrier medium preparation tool along with the harvesting tool 106. As the harvesting tool 106 passes through one opening 116 in the guidance grid 107, the blade in the carrier medium preparation tool pierces the carrier medium in the subsequent opening 116. When the harvesting tool 106 indexes to opening hole 116, the carrier medium in the current opening 116 has been pierced and the carrier medium in the subsequent opening 116 is pierced during that action. The pierce can be configured as a straight cut, X shape or other as desired, so that the coring tool 108 can pass through the slit created by the carrier medium preparation tool without removing any of the carrier medium. In another arrangement, a carrier medium preparation tool is configured as part of the harvesting tool 106. A blade fits within an opening in the extractor pin 111 and is actuated in a fashion that allows it to pierce the carrier medium prior to harvesting. Other methods of piercing the carrier medium are anticipated and within the scope of this innovation.

With reference to FIG. 4, the guidance grid 107 includes a generally flat, rectangular frame or structure 115 defining a plurality of openings 116 which are used as a guide for the harvesting tool 106 to harvest skin cores from a donor site. The guidance grid 107 can be configured to support the carrier medium by using two guidance grid sections 115 that are hinged or otherwise removably attached, with the carrier medium disposed between them. In this manner, the carrier medium is firmly supported around each opening through which the harvesting tool 106 passes as it harvests and transfers a core.

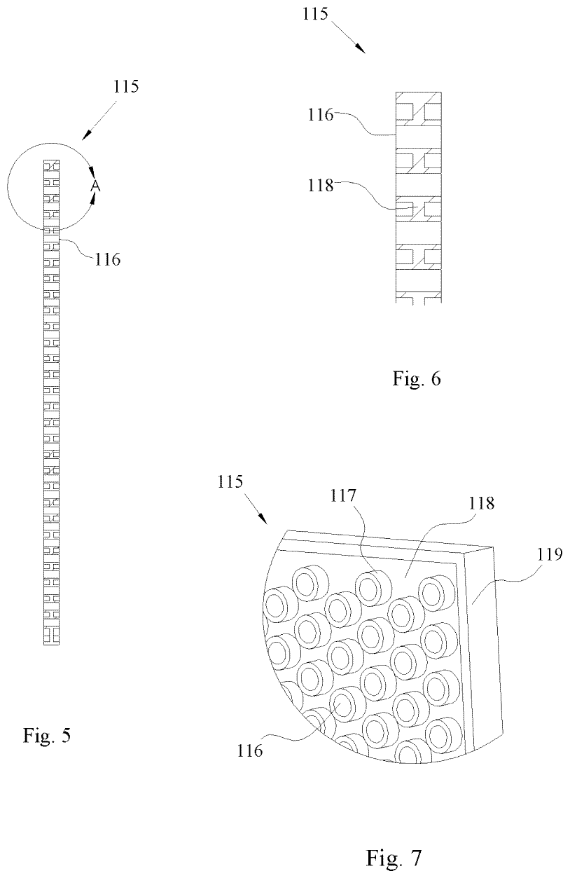

FIGS. 5 through 7 illustrate a guidance grid section 115. The illustrated arrangement is designed to support a carrier medium that is 102 mm wide and 127 mm high. This is a conventionally used size of a commercially available carrier medium. The grid section 115 defines a series of openings 116 arranged in a pattern as shown that provide a maximum density of, for example, 10%, resulting in 783 openings. That is, a 1.5 mm core harvested through each opening 116 results in 10% of the area of the skin tissue being removed from the donor site. Further, with the cores being disposed in the carrier medium, the resulting micro-core autograft can include a volume having a density of 10% skin cores.

In one arrangement, the thickness of the grid section 115 is 4 mm, and the inner diameter of the openings 116 provides clearance for a 2 mm diameter coring tool 108 to pass through and be guided by the opening 116. In this arrangement, the grid section 115 is constructed of cylindrical elements 117 and a central rib 118 to make the grid section 115 rigid as well as easy to be injection molded from plastic. A rim 119 defines the outer perimeter. Arrangements using different designs, materials or manufacturing techniques, use of alternative sizes of matrices or a quantity of openings 116 that produce higher or lower densities are anticipated and are within the scope of this innovation.

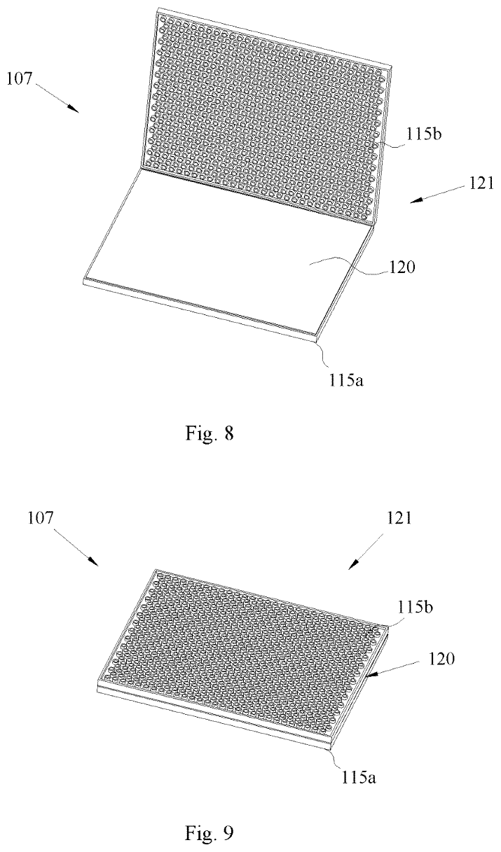

In one arrangement, with reference to FIGS. 8 and 9, the guidance grid 107 is constructed from two grid sections 115a, 115b. In one arrangement, opposing ends of the grid sections 115a, 115b are coupled via a hinge. FIG. 8 illustrates an isometric view of a hinged guidance grid 107 in an open position. A carrier medium 120 is disposed on the lower grid section 115a. FIG. 9 illustrates the guidance grid 107 in a closed position. The carrier medium 120 is disposed between the grid sections 115a, 115b where it is held and supported. The hinge 121 can be an integral part of the guidance grid 107, such as a hinge that is molded into the part at manufacture. Alternatively, the hinge 121 can be a separate part that is attached to the grid sections 115a, 115b. Other mechanisms can be used to attach the grid sections 115, such as a snap-together arrangement, etc.

As illustrated in FIG. 8, the carrier medium 120 is rectangular in shape having an area which substantially covers the lower grid section 115a. Such illustration is by way of example only. In one arrangement, the carrier medium 120 can be preconfigured in any shape. For example, a practitioner can cut the carrier medium 120 into a shape which corresponds to the shape of an area of a recipient site.

It may be desirable to have assistance in holding the guidance grid 107 against the patient's skin at the donor site during harvesting to provide stability during operation. In one arrangement, the surgical system 95 can include vacuum to secure the guidance grid 107 to a donor site. In other arrangements the guidance grid 107 may be strapped or otherwise held in place at a donor site.

FIGS. 10 and 11 illustrate a vacuum frame 122 that can be used with the surgical system 95. The vacuum frame 122 includes a flange 123 is configured to rest on the patient's skin at a donor site during use. The flange 123 may be of a rigid construction or may be flexible to conform to the patient. In some arrangements, the flange 123 can include an adhesive layer 124, such as double-sided tape, on the surface that contacts the patient. A vacuum port 125 is configured to connect to a vacuum source. A stepped wall 126 is configured to attach to a guidance grid.

FIG. 12 illustrates an isometric view of a guidance grid 107 having the vacuum frame 122 attached. In use, the adhesive layer 124 is exposed and the flange 123 is placed onto the patient's skin at the location of the donor site. A vacuum source (not shown) attaches to the vacuum port 125. A vacuum is applied to the space in the lower grid section 115a between the carrier medium 120 and the skin. The vacuum pulls both the carrier medium 120 and the skin against the grid section 115a, which stabilizes the guidance grid 107 during the harvesting procedure.

In one arrangement, when harvesting and transferring tissue cores from a donor site to the recipient site, the harvesting tool 105 is configured to harvest and transfer a set of tissue cores defining between about a 10% to 20% core density from the donor site to the recipient site. For example, with continued reference to FIG. 12, the design of the guidance grid 107 defines the maximum quantity of cores that can be harvested and transferred to the carrier medium. Quantities lower than the maximum may be achieved by selectively omitting openings. The core density in the carrier medium is determined by the number and placement of cores and the diameter of each core. Core density is defined as the percentage of skin core area in the populated area of the carrier medium. Multiple factors can contribute to the choice of core density as well as the diameter of cores.

In general, a relatively higher core density can result in more rapid and complete healing at the recipient site. However, the relatively higher core density requires more cores or larger cores per unit area. An increase in the number of cores harvested can increase the time it takes to harvest the cores and can cause additional trauma at the donor site. It can be advantageous to use the minimum core density that can result in substantially complete healing. Clinical studies have determined that a core density of about 10% produces an optimal tradeoff. At a 10% core density, the cores grow sufficiently to fill in the recipient site in three to four weeks. Less than 10% leaves gaps in the skin, resulting in scarring, extended healing times and less optimal healing outcomes. Increasing the density can have a relatively minor impact on healing and may result in lengthier donor surgeries and increased scarring at donor sites. A core density of 20% is approximately the maximum core density value, above which wound site healing is no longer improved and the donor site is more significantly damaged.

In one arrangement, with reference to FIGS. 2 and 3, the coring tool 108 is configured as a hollowed, generally cylindrical shaft having a sharpened cutting edge 109 disposed at a distal end and a core cutting diameter 204 configured to create and extract tissue cores having a diameter of between about 1.0 mm and 3.0 mm. The diameter of the core used in this innovation can be chosen based on certain factors concerning both the recipient site and the donor site. One factor relates to the presence of skin adnexa in the cores. During operation, the harvesting tool 106 is configured to harvest a core that contains the skin adnexa, for example, hair follicles, sweat glands and sebaceous glands. The presence of adnexa in the core allows the skin at the recipient site to heal with a normal skin appearance and function, such as sensation, contractility, lubrication, and heat loss. It can be difficult to successfully harvest cores of 1 mm or less in diameter. The relatively small size of these cores makes them fragile and difficult to remove intact. Further, harvesting cores of 1.0 mm or less in diameter does not capture skin adnexa.

A second factor in determining core diameter is the quantity of cores necessary to produce a desired core density within a micro-core autograft. Increasing core size exponentially increases core area. For example, increasing the diameter from 1.0 mm to 1.5 mm increases the surface area of the core by 225% which reduces the number of cores harvested from the donor site to approximately less than half.

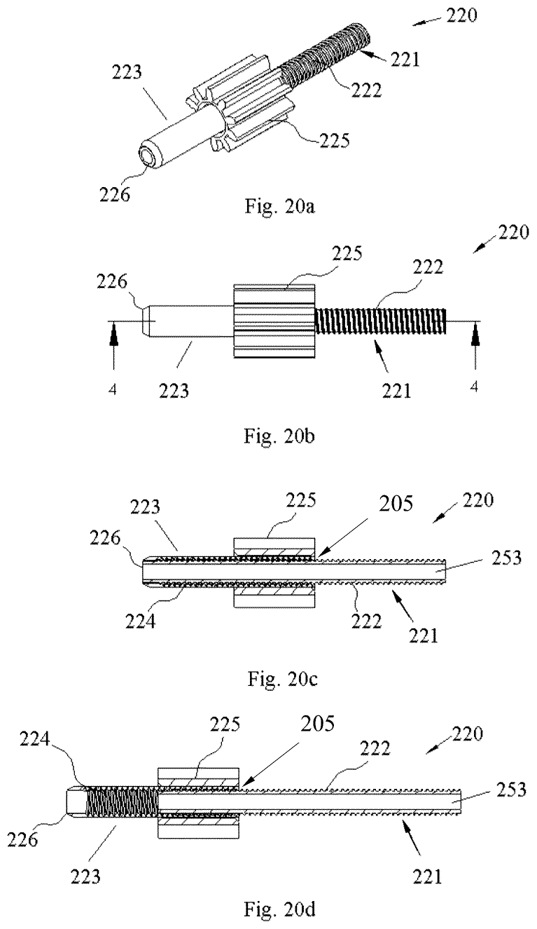

A third factor in determining core diameter is the effect of core size on healing at the donor site. Diameters greater than 1.5 mm have an increased risk of causing scars or blemishes, depending on the patient's age, gender and health. Once the core diameter approaches 2.0 mm, the donor site can begin to have difficulty healing completely, leaving visual scarring and other negative effects. In some patients, however, such as young healthy males, cores of up to 3.0 mm can be harvested and still result in near normal skin function and appearance at the donor site. The micro-coring system 100 can be configured to harvest cores of a diameter to suit specific patient types. A core diameter of 1.5 mm can be used with all patient types without a negative effect. In one arrangement, the core diameter is 1.5 mm with a density of 10% resulting in a total of 783 cores in a 102 mm×127 mm carrier medium. Additionally, the core depth may be configured to extend into the donor tissue depending on factors such as tissue type, age and condition of the patient, etc. Typically, core depth is in the range of between about 2 mm and 5 mm. The shape of the coring tool 223, as shown for example in FIG. 20a as round, may be configured with different shapes, such as elliptical, square, etc. if so desired.

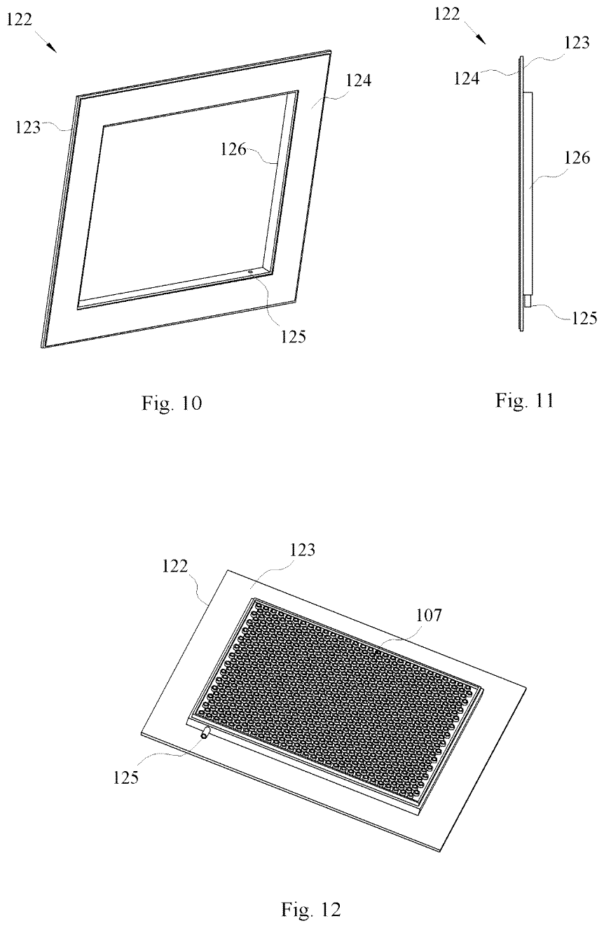

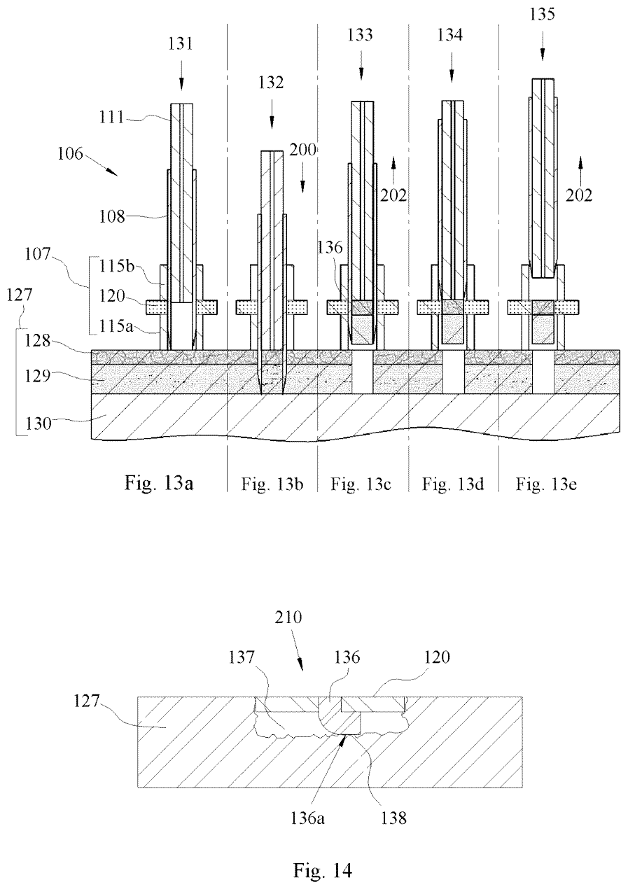

FIGS. 13a through 13e illustrate an example of a method for harvesting a core in one arrangement of the current innovation. Each of FIGS. 13a through 13e illustrates a cross sectional representation of the skin tissue 127, a harvesting tool 106 having a coring tool 108 and extractor pin 111, the guidance grid 107 having the upper and lower grid sections 115a, 115b, and the carrier medium 120. The epidermis 128 is the outermost protective skin layer. The dermis 129 includes hair follicles, sweat glands, sebaceous glands, nerve endings, blood vessels, lymph vessels, etc., held together by collagen. The subcutis 130, the innermost layer of skin, includes a network of fat and collagen cells.

As shown in FIG. 13a, in a first position 131, the coring tool 108 is disposed through the guidance grid 107, where it has penetrated the carrier medium 120 and is flush with the top of the epidermis 128. The extractor pin 111 is disposed in a retracted position from the epidermis 128 by the desired core length.

As shown in FIG. 13b, in a second position 132, the coring tool 108 has moved along direction 200 and has penetrated the epidermis 128 and dermis 129. The extractor pin 111 is flush with the top of the epidermis 128. In one arrangement, the coring tool 108 can penetrate a small distance into the subcutis 130 as the subcutis fat layer eases the separation of core. This can ensure that all of the dermis has been harvested. Taking a small portion of the subcutis 130 can also help to capture stem cells that can be located at the dermis 129 to subcutis 130 boundary. At this point, the application of vacuum to the extractor pin 111, or another method may be used to help the core adhere to the extractor pin 111.

As shown in FIG. 13c, in a third position 133, the coring tool 108 has captured the core 136 and has moved along direction 202 to a retracted position in which the top of the epidermis 128 within the core 136 is even with the top of the carrier medium 120.

As shown in FIG. 13d, in a fourth position 134, the extractor pin 111 is held in place and the coring tool 108 has been retracted past the carrier medium 120. The core 136 is now fully captured within the carrier medium 120. At this point the vacuum, if used, can be vented, allowing the core 136 to release from the extractor pin 111.

As shown in FIG. 13e, in a fifth position 135, the harvesting tool 106 has further moved along direction 202 to a retracted position from the carrier medium 120. Once the harvesting tool 106 exits the guidance grid 107, it is ready to be indexed along a lateral direction relative to the guidance grid 107 to harvest the next core 136.

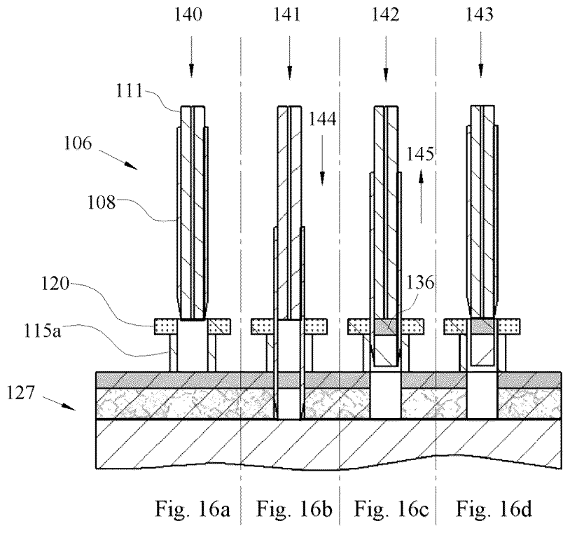

In another arrangement, the extractor pin 111 is configured to remain stationary with respect to vertical longitudinal (Z) axis during operation. For example, FIGS. 16a through 16d illustrates skin tissue 127, the harvesting tool 106, as well as the lower grid section 115a and the carrier medium 120. The upper grid section is not used since the extractor pin 111 does not move vertically in this arrangement. This arrangement can be configured to be utilized with a manufactured scaffolding that is rigid or semi-rigid. For clarity, the scaffolding is not shown in FIGS. 16a through 16d.

As shown in FIG. 16a, in a first position 140, the harvesting tool 106 is disposed above the carrier medium 120 with a bottom of the coring tool 108 and the extractor pin 111 substantially even with a top surface of the carrier medium 120. As shown in FIG. 16b, in a second position 141, the coring tool 108 has moved downward along direction 144 and penetrated the skin tissue 127 to the predetermined depth in order to capture a core 136. The extractor pin 111 remains stationary relative to the top surface of the carrier medium 120. As shown in FIG. 16c, in a third position 142, the coring tool 108 has moved upward along direction 145 until the top of the core 136 is disposed in proximity to or has made contact with the extractor pin 111. At this point, the core 136 can remain in place with the top of the core 136 even with the top surface of the carrier medium 120. As shown in FIG. 16d, in a fourth position 143, the coring tool 108 has moved upward along direction 145, back into its starting position. The core 136 stays in place in the carrier medium 120. The extractor pin 111 has remained stationary relative to the longitudinal (Z) axis throughout the process. The harvesting tool 106 is now ready to advance to the next core harvesting location.

The depth that the coring tool 108 penetrates the skin can depend upon gender, age and health condition of the patient, as well as the location on the patient's body of the selected donor site. Depending on these factors, the penetration depth can range from between about 1 mm to about 5 mm. The micro-coring system 100 has the capability for selectable penetration depths. Thickness of skin is well understood in the art. The surgeon using the micro-coring system 100 can select the penetration depth based upon the above referenced factors.

As can be seen in FIG. 13c, the captured core 136 extends below the carrier medium 120. This positioning can aid in healing when the micro-core autograft (i.e., populated carrier medium) has been placed onto the recipient site.

For example, FIG. 14 illustrates a cross sectional side view of a wound with a micro-core autograft 210 applied. Only one core 136 is shown for clarity. The skin tissue 127 contains a wound area 137. A micro-core autograft 210 (i.e., a carrier medium 120 with core 136) has been applied. Since the core 136 extends below the carrier medium 120, it curves within the wound area 137 as shown. The horizontal portion 136a of the core 136 makes an extended contact 138 with the wounded skin tissue. This has been shown to increase the rate of tissue growth and healing. It is believed that the enlarged contact area 138 between the core 136 and the skin tissue 127 allows more cells to attach and grow than if only the end of the core 136 made contact.

The following is an example sequence of operation for an arrangement of the micro-coring system 100.

The micro-coring system 100 is removed from its sterile packaging. A battery isolating pull tab activates the device. A practitioner enters specific parameters through, for example, a computerized device such as a smart phone executing a skin core harvesting application, a tablet device, or USB device. The parameters can be preset parameters or can be selected by the practitioner at the beginning of the process. While the parameters can be configured in a variety of ways, in one arrangement, the parameters determine the depth of the core 136, the quantity of cores 136 to be harvested, and the pattern of harvesting cores 136, as well as other selectable inputs.

Next, a carrier medium 120 is installed within the guidance grid 107. The cover strip is removed from the double-sided tape 124 on the flange 123 of the vacuum frame 122. The guidance grid 107 of the micro-coring system 100 is placed on the selected donor site. Upon an input from the surgeon, vacuum begins and the guidance grid 107 and the micro-coring system 100 are held in place on the donor site.

Next, the skin core harvesting process begins. The harvesting tool 106 is indexed to its starting position. If so equipped, a carrier medium preparation tool pierces the carrier medium 120 through an opening 116 in the guidance grid 107. The harvesting tool 106 now extends through this opening 116 and harvests a core as illustrated in FIG. 13a. Once the core 136 has been harvested and captured in the carrier medium 120, the harvesting tool is retracted and indexed to the next opening 116 in the guidance grid 107. This process continues until harvesting is complete. The vacuum is now released and the guidance grid 107 and micro-coring system 100 is removed from the patient. The guidance grid 107 is opened and the resulting micro-core autograft is removed from the grid 107 and placed on the recipient site.

The cores 136 within the micro-core autograft retain their orientation, both vertically and axially. The surgeon can place the micro-core autograft on the recipient site and orient its direction in a manner that helps to promote and preserve normal skin function, such as elasticity, hair growth and other attributes.

The micro-coring system 100 can harvest a fully populated carrier medium, for example, a quantity of 783 1.5 mm diameter cores 136 in a 102 mm×127 mm carrier medium 120.

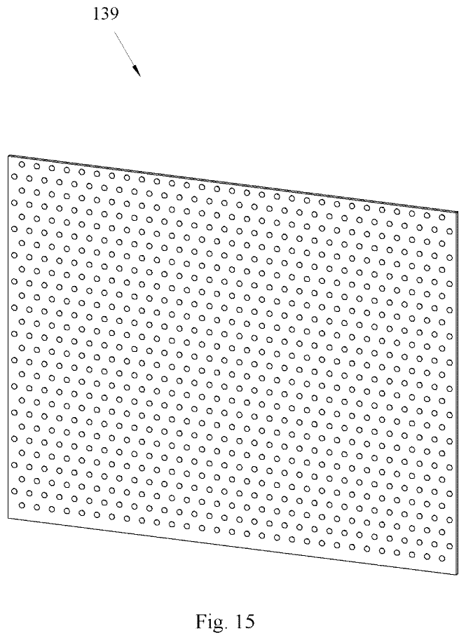

FIG. 15 illustrates an isometric view of a micro-core autograft 139 of this arrangement. The system 100 also has extended capabilities, such as populating only a partial area of the grid, which is useful when the recipient wound area is smaller than an entire grid. Lower core density is possible if a lower seed density is desired by, for example, harvesting a core from every other opening for a density half of a standard grid. Cores may be harvested in a pattern that produces a predetermined shape, such as a shape which mirrors the shape of the wound 137, which is useful for tailoring a graft to a specific recipient wound.

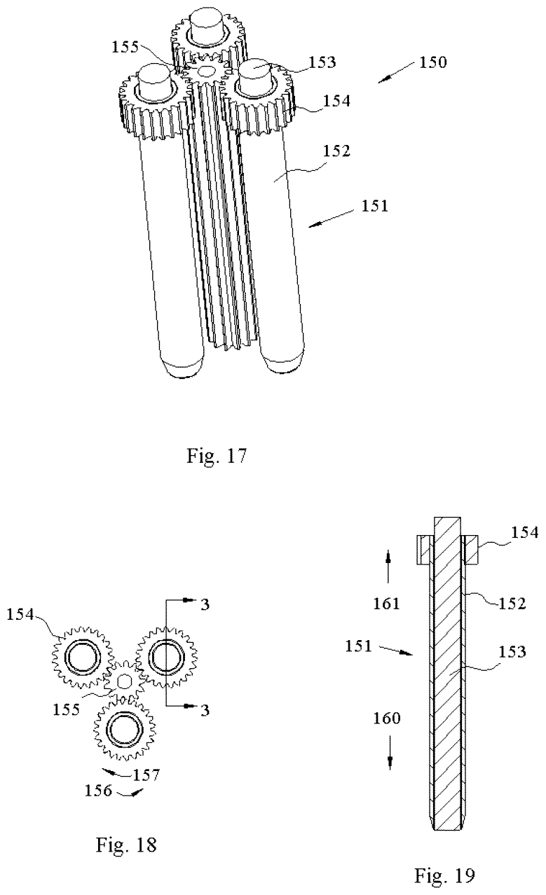

In one arrangement, the micro-coring system 100 can be configured to harvest multiple cores from skin tissue 127 at a single time, thereby increasing efficiency and reducing the time needed to prepare a carrier medium. For example, FIGS. 17 through 19 illustrate an arrangement of a harvesting assembly 150 having three harvesting tools 151 disposed in a triangular arrangement and driven by a common drive mechanism, such as a rotary drive mechanism.

As illustrated, each harvesting tool 151 includes a coring tool 152 and an extractor pin 153. Affixed to the top of each coring tool 152 is a driven gear 154. The inner diameter of the coring tool 152 defines a female screw thread (not shown). The outer diameter of the extractor pin 153 defines a male screw thread configured to mesh with the female screw thread of the coring tool 152. The coring tool 152 and extractor pin 153 may be threaded along their entire lengths or for a portion of their lengths with a clearance slip fit on the remainder of their lengths. In one arrangement, the drive mechanism is configured as a pinion shaft 155 located centrally relative to the three harvesting tools 151 and is meshed with each of the three driven gears 154. A drive source (not shown) is configured to rotate the pinion shaft 155 in both a clockwise and counterclockwise direction. The three harvesting tools 151 and pinion shaft 155 are held in their relative axial positions by a structure (not shown) that is configured to translate the harvesting assembly 150 in the X and Y directions to locate the harvesting assembly 150 at a desired position above the skin tissue 127. Additionally, the structure is configured to hold the extractor pin 153 in a stationary longitudinal (Z axis) position relative to the skin tissue 127.

In operation, the structure (not shown) moves the harvesting assembly 150 to a predetermined position above the skin tissue 127. The pinion shaft 155 is made to rotate by the drive source (not shown) in, for example, a counterclockwise 156 direction. The pinion shaft 155 rotation causes the three driven gears 154 to rotate in a clockwise direction 157, thus rotating the coring tools 152. Since the coring tools 152 are threaded onto the fixed extractor pins 153, the rotation causes the coring tools 152 to move in a downward direction 160. The coring tools 152 then penetrate the skin tissue 127 to a desired depth to capture three cores 136. The inherent spinning action of the descending coring tools 152 aids in cutting the tissue 127 as the coring tools 152 penetrate the skin tissue 127. Once the cores 136 are harvested, the pinion shaft 155 is made to rotate in the opposite (clockwise) direction 157, rotating the coring tools 152 in a counterclockwise direction 156 and causing them to move in an upward direction 161. When the cores 136 contact a bottom portion 158 of the extractor pin 153, they are held in place and become captured within the carrier medium 120 while the coring tools 152 retract to their starting position. The harvesting assembly 150 can then be relocated to the next harvesting position.

While the above example utilizes three harvesting tools 151 and one pinion shaft 155, in another example, multiple harvesting assemblies 150 can be included in one micro-coring system 100. Other drive arrangements may include, for example, a pinion shaft 155 that drives one or more harvesting tools 151, wherein adjacent harvesting tools are geared together so that rotation of one harvesting tool 151 drives the adjacent one.

FIGS. 20a through 20d illustrate an arrangement of a harvesting tool 220. As illustrated, the harvesting tool 220 includes an extractor pin 221 defining an external screw thread 222 and a coring tool 223 defining an aperture 205 extending along a longitudinal axis of the coring tool 223 and an internal screw thread 224 which mates with the external screw thread 222 of the extractor pin 221. The coring tool 223 is configured to include a sharpened edge 226 disposed at a distal end that is configured to penetrate tissue at a donor site and capture a tissue core from the tissue. The coring tool 223 can include an attached gear 225 disposed on an outer surface of the coring tool 223, which, when driven, is configured to rotate the coring tool 223 and cause the coring tool 223 to translate (e.g., extend from, or retract), along a longitudinal axis of the extractor pin 221. In operation, the extractor pin 221 remains stationary relative to the skin tissue, and the gear 225 is driven by a motor, another gear, or other mechanism, for example. The rotation of the threads 224 of the coring tool 223 around the threads 222 of the stationary extractor pin 221 causes the translation (e.g., extension and retraction) of the coring tool 223 relative to the extractor pin 221. For example, as shown in FIG. 20c, the coring tool 223 is first disposed in a retracted position relative to the extractor pin 221. In the retracted position, an operator can place the harvesting tool 220 against a donor site tissue. Rotation of the coring tool 223 relative to the stationary extractor pin 221 in a first direction positons the coring tool 223 in a first extended position as shown in FIG. 20d. As illustrated, in the extended position, the extractor pin 221 is fully inserted into the coring tool 223 and the coring tool 223 cuts through a portion donor site tissue such that it captures or harvests a tissue core from the site. Following capture of the core by the coring tool 223, the operator can manually lift the harvesting tool 220 from the donor site. Rotation of the coring tool 223 relative to the stationary extractor pin 221 in a second direction (i.e., where the second direction is opposite to the first direction of rotation) causes the coring tool 223 to translate to a second retracted position relative to the extractor pin 221, as is shown in FIG. 20c. With such translation, the extractor pin 221 extracts the core from the harvesting tool 220 and places the core onto a secondary location, such as onto either a carrier medium on into a recipient site.

With this arrangement, as the coring tool 223 is extended, a sharpened end 226 of the coring tool 223 rotates as it penetrates the tissue at the donor site. This rotation creates a shearing force between the coring tool 223 and donor site that aids in cutting the tissue by lowering the amount of penetration force required to cut the tissue. It also creates a relatively clean cut and causes less trauma to the tissue as it cuts. The coring tool 223, with the harvested core, is pulled away from the donor site tissue, and the harvested core remains inside the coring tool 223. The harvesting tool 220 is then moved to a carrier medium or directly to a wound site. When in position, the drive direction of the gear 225 can reverse, and the extractor pin 221 holds the core in place as the coring tool 223 is retracted, causing the core to exit the coring tool 223 and be placed at the desired location.

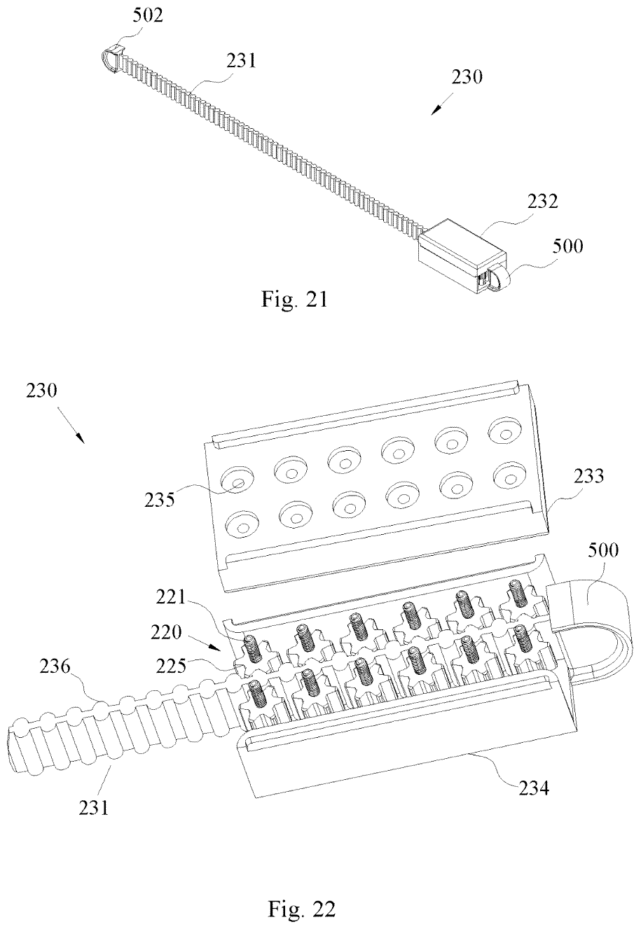

FIG. 21 shows an arrangement of a micro-coring device 230 that can utilize multiple harvesting tools 220 in order to harvest multiple cores with each actuation. In one arrangement, the micro-coring device is configured as a manual device. During operation, a user can manually actuate a pull strip 231 relative to a device body 232 to rotate gears 225 carried by the device body 232 to actuate corresponding harvesting tools 220.

FIG. 22 is a close-up isometric view of the micro-coring device 230 in which an upper section 233 of the body 232 has been removed from the lower section 234 and turned upside down in order to see its underside and the inside of the micro-coring device 230. In this arrangement, there are twelve harvesting tools 220 that are operated simultaneously when the pull strip 231 is pulled from one end of its travel to the other. The recesses 235 in the upper section 233 of the body 232 are configured to guide and longitudinally retain each of the harvesting tools 220. For example, each of the extractor pins 221 can be retained in a corresponding recess 235 in a manner that mitigates translation relative to the body 232. The gears 225 mesh with ribs 236 defined by the pull strip 231. Longitudinal motion of the pull strip 231 causes the ribs 236 to rotate each gear 225 which, in turn, causes each corresponding coring tool 223 to either extend downward or retract upward. The harvesting tools 223 on one side of the pull strip 231 can be configured with right hand threads, while the harvesting tools 223 on the opposite side of the pull strip 231 can be configured with left hand threads, so that the gears 225 on either side of the pull strip 231 rotate in opposite directions. In this manner, all the coring tools 223 extend and retract in unison.

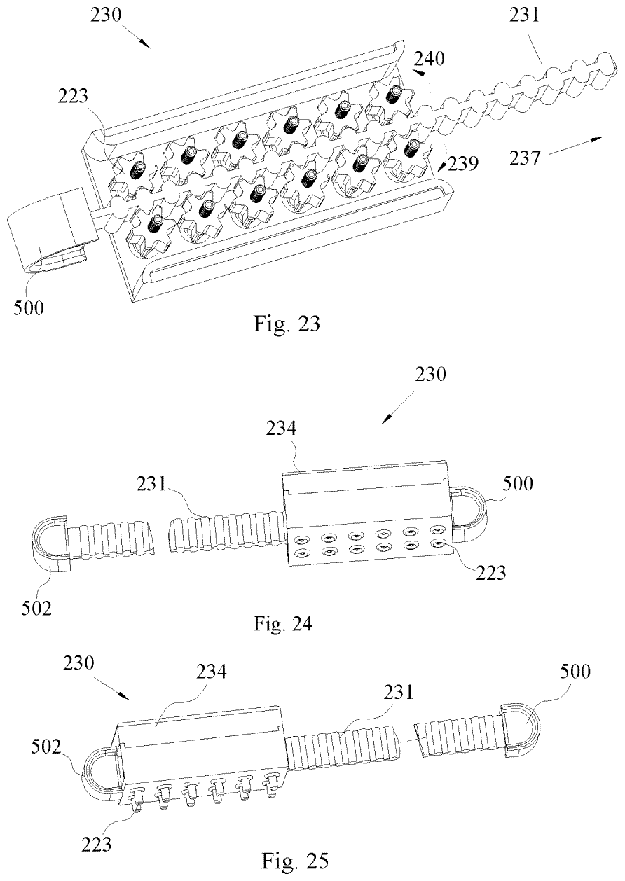

FIG. 23 shows an isometric view of the micro-coring device 230 with the top 233 of the body 234 removed for clarity. In this view, the pull strip 231 (partially shown) is being actuated in a direction 237 from left to right. At each end of the pull strip 231 there is a handle 238 that is configured to be grasped by a thumb and forefinger of an operator's hand. When pulled, the pull strip 231 causes the coring tools 223 that are disposed above the pull strip 231 rotate in a counterclockwise direction 239, and the ones below the pull strip 231 rotate in a clockwise direction 240. In this example, if the harvesting tools that are above the pull strip 231 have left handed threads, and the ones below have right handed threads, actuation of the pull strip 231 to the right 237 causes the coring tools 223 to extend, and actuation of the pull strip 231 to the left causes them to retract.

FIG. 24 shows an isometric view from below of the micro-coring device 230 with the pull strip 231 (partially shown) actuated to the left and the coring tools 223 retracted. With such positioning, a first end 500 of the pull strip 231 is disposed in proximity to the body 234. FIG. 25 shows an isometric view from below of the micro-coring device 230 with the pull strip 231 (partially shown) actuated to the right and the coring tools 223 extended. With such positioning, a second end 502 of the pull strip 231 is disposed in proximity to the body 234. This arrangement of the micro-coring device 230 can harvest and deliver twelve cores with each operation.

In use, the micro-coring tool 230 is placed and held onto the skin at the donor site, then the pull strip 231 is pulled to extend the coring tools 223 into the donor tissue. The micro-coring device 230 is then lifted from the donor site, retaining the cores inside the coring tool 223. The micro-coring tool 230 is then moved either to a carrier medium or directly to the wound site. The pull strip 231 is then pulled in the opposite direction, causing the cores to be extracted and placed onto the carrier medium or donor site. Other arrangements of this innovation may have more or less harvesting tools 220 as desired and still function in the same manner.

A pull strip 231 may be in the form of a continuous loop (not shown) that passes through the micro-coring device 230. In this arrangement, a user can grasp a first portion of the continuous loop and pull it in a first direction, and can then grasp a second portion of the continuous loop and pull it in the first direction, repeating the action until the coring tools 223 are extended or retracted. In some arrangements, the continuous pull strip may be completely enclosed in the body 234 of the device, where it is configured to be driven by a motor or other drive system in order to automate extension and retraction.

Other arrangements of harvesting tools 220 are within the scope of this innovation. Other formats of layout, pull strip design, etc. are envisioned and are within the scope of this innovation.

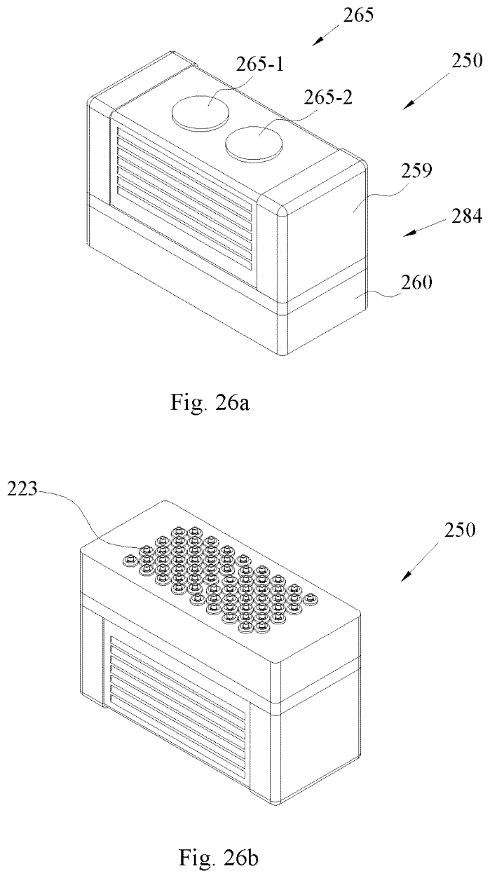

FIGS. 26a through 28 illustrate an arrangement of a micro-coring device 250 having multiple automated harvesting tools 220. In this arrangement, sixty-four harvesting tools 220 can be disposed in an array of eight staggered rows having eight harvesting tools 220 each. In one arrangement, this array may be approximately 25 mm by 50 mm. For example, in the case where each coring tool 223 has a diameter of about 1.5 mm, this results in the micro-coring device 250 harvesting approximately 10% of the tissue of the donor site area that the micro-coring device 250 covers.

With reference to FIG. 28, the harvesting tool 220 of the micro-coring device 250 can be configured with independent harvesting tool portions 280, 282. For example, the harvesting tool 220 can include a gear array 221 divided into two portions along line 261. With such an arrangement, a first set of the gears 225 of the gear array 221 (e.g., on a first side of the line 261) do not mesh with a second set of the gears 225 of the gear array 221 (e.g., on a second, opposing side of the line 261). For each of the first and second set of gears, the gears 225 of each row are meshed with the gears 225 in the next row. When a gear 225 in one set is rotated, all the gears 225 in that set can rotate. The gears 225 in each horizontal row drive their mating gears 225 in the opposite direction, so horizontal rows can alternate between left and right handed threads so that the coring tools 223 extend and retract together.

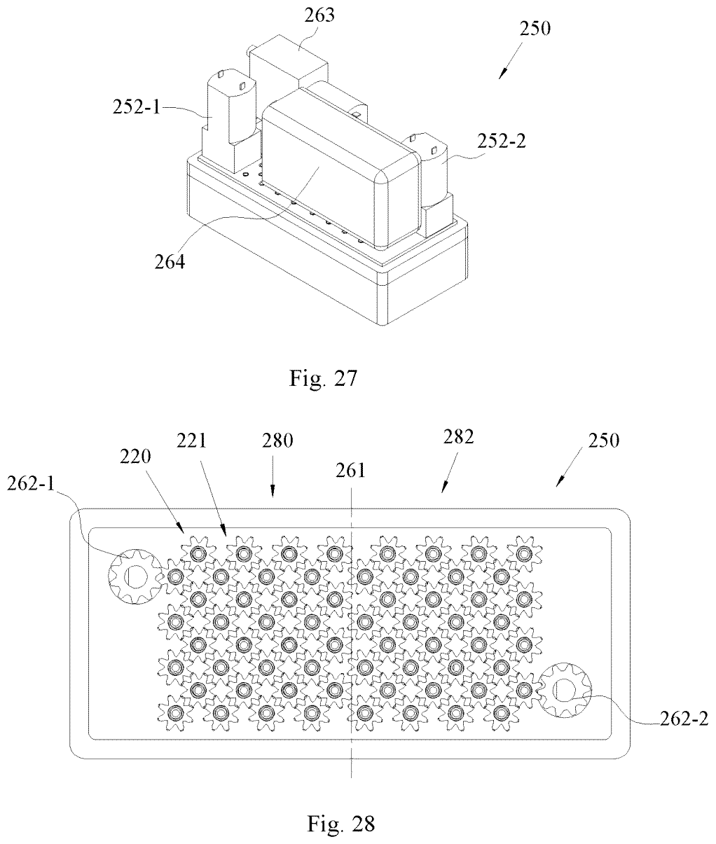

As shown in FIG. 27, the micro-coring device 250 can include one or more electric motors 252 disposed in operative communication with the gears 225. For example, the micro-coring device 250 can include a first motor 252-1 having a first drive shaft and a second motor 252-2 having a second drive shaft. Each motor drive shaft can have a corresponding gear 262-1, 262-2 mounted thereon and mesh with one or more corresponding gears 225 of the harvesting tool portions 280, 282. During operation, as the first and second drive shafts rotate, the motor gears 262-1, 262-2 drive the gears 225 that in turn rotate the coring tools 223. The mating threads of the harvesting tools 220 cause the coring tools 223 to extend or retract. In one arrangement, the length of each of the motor gears 262-1, 262-2 is configured retain a mesh with the corresponding gears 225 of the harvesting tool portions 280, 282 as the coring tool 223 extends and retracts.

In one arrangement, two motors 252-1, 252-2 can be used in order to mitigate the effect of any backlash between gears 225 that may accrue and cause the coring tools 223 to extend and retract different distances. For example, if only the first motor 252-1 is operated, only half of the coring tools 223 (e.g., the coring tools 223 associated with the first harvesting tool portion 280) can be extended. This may be advantageous when a relatively smaller, in this example 25 mm×25 mm and 32 cores, array corresponding to the size and shape of the wound to be treated is selected. This example gear and motor arrangement illustrates the function of the micro-coring device 250 and is not meant to be limiting. Other arrangements with different quantity or pattern of harvesting tools 220 or quantity and arrangement of motors 252 are within the scope of this innovation.

In some arrangements, the gears 225 may be replaced by elastomeric rollers (not shown). Each roller can be disposed in an interference relationship to an adjacent rollers. For example, the harvesting tools 220 may be spaced apart by 4 mm, and the diameter of each roller may be 4.2 mm. Since the rollers can include an elastomeric material, two adjacent rollers press into each other with approximately 0.4 mm of interference. In this manner, when one elastomeric roller is rotated, friction between adjacent rollers causes those adjacent rollers to rotate, thereby extending and retracting all associated coring tools 223 simultaneously. The actual diameter of the rollers, the relative hardness of the rollers, and the spacing of the rollers can be determined by desired coring tool 220 spacing and specific roller drive characteristics.

With reference to FIG. 27, in one arrangement, the micro-coring device 250 can include a vacuum pump 263 disposed inside a housing 284 of the micro-coring device 250. The vacuum pump 263 can be configured to create a vacuum between a top housing 259 and a bottom housing 261 of the micro-coring device 250. For example, with additional reference to FIGS. 20c and 20d, the extractor pin 221 of each harvesting tool 220 can define a central aperture 253 (FIGS. 20c, 20d) that runs the length of its longitudinal axis and that is configured to be disposed in fluid communication with a vacuum source, such as the vacuum pump 263. During operation, the vacuum pump 263 can create a vacuum in the aperture 253 of each extractor pin 221 and within the hollow coring tool 223, thereby forming a vacuum at a sharpened end 226 of each coring tool 223. When the micro-coring device 250 is placed onto a donor site, this vacuum helps to keep the micro-coring device 250 in place as core harvesting is performed. The vacuum also aids in harvesting a core, as it can help to hold the core inside the coring tool 223 as the tool 223 is removed from the donor site.

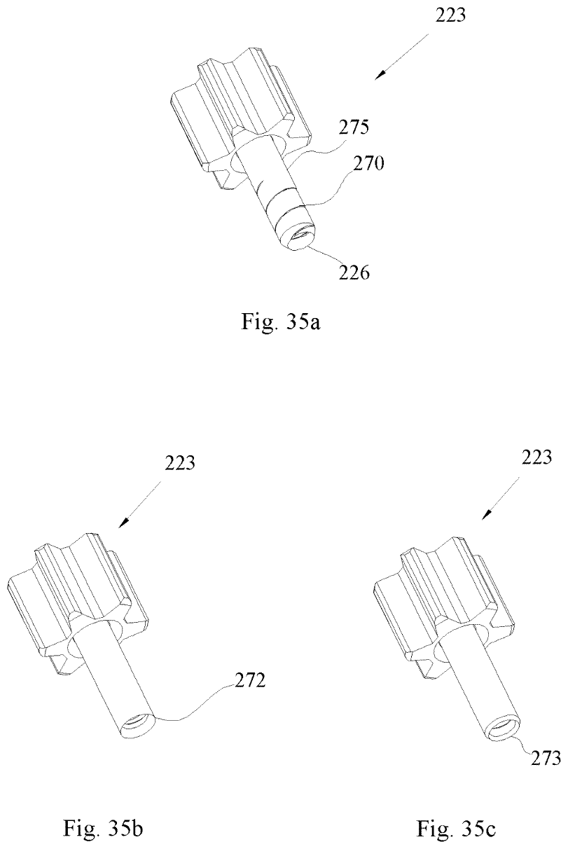

FIG. 35a illustrates an isometric view of a coring tool 223 with an additional feature that aids in holding the core in the coring tool 223 as the coring tool 223 is removed from the donor site tissue. For example, the coring tool 223 can include a spiral slot 270 which has been cut into a coring tool wall 275 and which extends between the sharpened edge 226 and a distal location relative to the sharpened edge 226. As such, the coring tool wall 275 is configured as a coiled spring. The spiral slot 270 is cut in a direction so that as the coring tool 223 is rotated and advanced into the tissue, the coils of the coring tool wall 275 unwind, thereby making the diameter of the coring tool 223 larger than in an unused state. During operation, as the coring tool 223 is advanced into the tissue, the spiral slot 270 is configured to cause the coils of the coring tool wall 275 of the coring tool 223 wall to unwind to increase the diameter of the sharpened end 226 relative to the tissue, thereby creating a core having a relatively larger inside diameter. As illustrated, a right handed thread can rotate the coring tool 223 in a direction to facilitate this. For a left handed thread, the rotation of the spiral 270 can be cut into the coring tool 223 in the opposite direction. When the coring tool 223 stops rotating and advancing, the slot 270 causes the coring tool wall 275 of the coring tool 223 radially retract, such as a spring, and clamp onto the core (e.g., the coring tool wall 275 springs back to its original size and holds the core in place). If needed, the coring tool 223 can be driven in reverse a small amount in order to aid the spring action. The core can be ejected from the coring tool 223 in the same manner as described above.

In the arrangement of FIG. 35a, the end of coring tool 223 is sharpened from an outer diameter towards an inner diameter, placing the sharpened point 226 on its inner diameter. This arrangement cuts a core that is equal in diameter to the inner diameter of the coring tool 223. FIGS. 35b and 35c illustrate isometric views of coring tools 223 with differing sharpening arrangements that aid in holding the core in the coring tool 223 as the coring tool 223 is removed from the donor site tissue.

In the arrangement of FIG. 35b, the end of the coring tool 223 is sharpened from the inner diameter towards the outer diameter, placing the sharpened end 272 on the outer diameter of the coring tool 223. In this manner, the core is cut with a diameter equal to the outside diameter of the coring tool 223 rather than the inside diameter. The cut core is larger than the inside diameter of the coring tool 223 creating a compression as the core advances into the coring tool 223. This compression causes the core to apply pressure to the inner diameter of the coring tool 223 which tends to hold the core in place as the coring tool 223 is withdrawn from the donor site. In the arrangement of FIG. 35c the end of the coring tool 223 is sharpened from both the outer diameter and the inner diameter, resulting in the sharpened point 273 being between the outer diameter and the inner diameter. This arrangement cuts a core with a diameter that is larger than the FIG. 35a arrangement, but smaller than the FIG. 35b arrangement resulting in a compressive force that is less than the FIG. 35b arrangement. In application, the location of the sharpened point and therefore the cut diameter of the core can be selected to produce a desired amount of compression of the core inside the coring tool 223 and the resultant amount of the tendency to hold the core within the coring tool 223 when the coring tool 223 is withdrawn from the donor site.

Returning to FIG. 26a, the micro-coring device 250 can also include an electronic controller (not shown) capable of operating the drive motors 252-1, 252-2 and vacuum pump 263. A user interface can also be included with the micro-coring device 250. For example, with reference to FIG. 26a, the micro-coring device 250 can include an interface such as actuators 265-1, 265-2 on the top of the device 250. During operation, a user can depress a first actuator 265-1 to extend the coring tools 223 and can depress a second actuator 265-2 to retract the coring tools 223. In some arrangements, the interface may be configured as a touch screen or other device attached to the micro-coring unit 250, or it may be an application on an external computerized device such as a smartphone or tablet, for example. Based on parameters input by an operator, the interface can drive the motors 252 to extend the coring tools 220 to a desired depth and apply vacuum when desired. The electrical power used to operate the drive motors 252-1, 252-2 and vacuum pump 263 may be provided by an internal battery 264 or external power source.

Once the cores are extracted from a tissue location, they may be placed directly onto the wound site. Alternately, the cores may be placed onto a carrier medium and the carrier medium containing the harvested cores then placed onto the wound site.

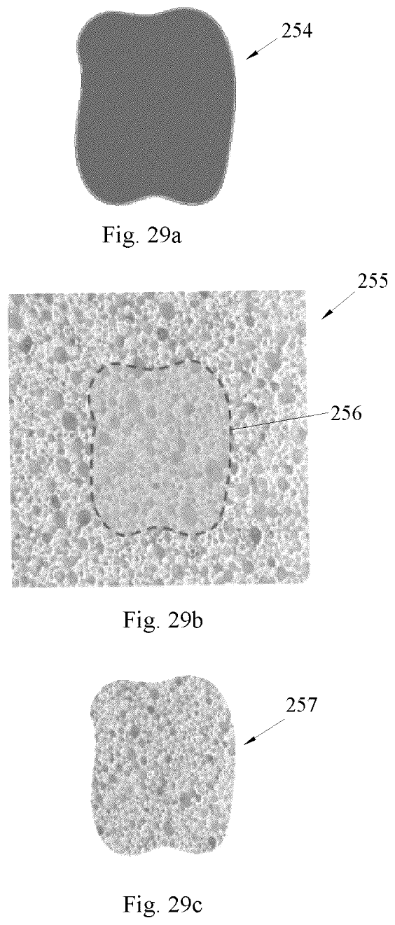

FIGS. 29a through 34 illustrate an example core placement process using the micro-coring device 250 in conjunction with a core manager system 260.

At the start of the process, a donor site and a wound site are prepared as needed for grafting. A carrier medium is then cut to the size and shape of the wound. FIG. 29a depicts an example of the shape of the damaged tissue at the wound site 254. A template is made of the wound site via tracing or other method. FIG. 29b shows a carrier medium material 255 with the template 256 of the shape of the wound disposed thereon. The carrier medium is cut to the shape defined by the template 256. The resulting carrier medium 257 as removed from the carrier medium material 255 is illustrated in FIG. 29c. Following removal, the carrier medium 257 conforms to the shape and size of the wound to be treated at the wound site 254.

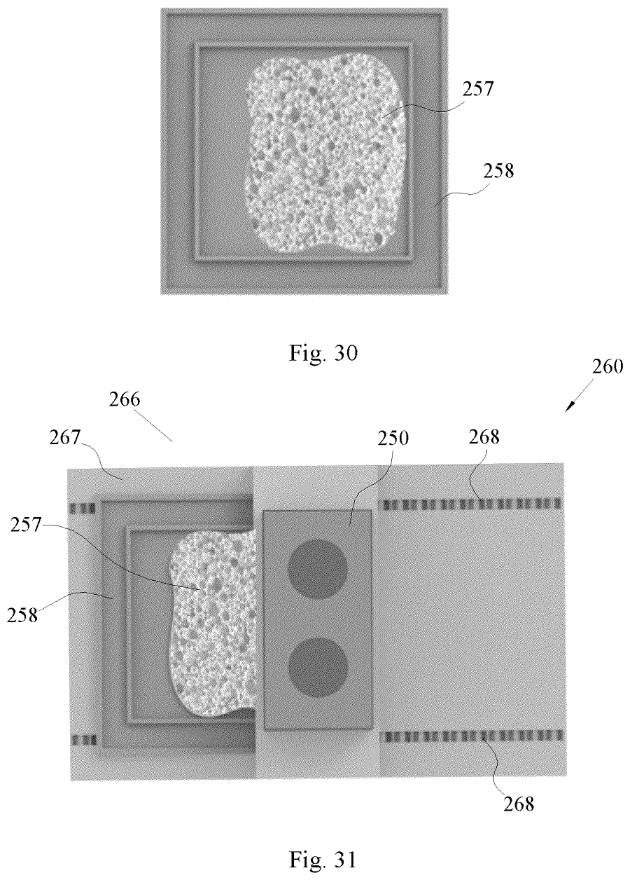

Following preparation of the carrier medium 257, the carrier medium 257 is placed in a core manager system 260 configured to position the carrier medium 257 relative to a micro-coring device 250 to facilitate core population. For example, FIG. 30 illustrates a plan view of the carrier medium 257 that is placed onto a core manager tray 258 of the core manager system 260 and FIG. 31 is illustrates a plan view of a core manager 266 of the core manager system 260 that carries the core manager tray 258. The core manager 266 includes a base 267 having guides 268 onto which the core manager tray 258 is placed. The micro-coring device 250 can be placed into a fixed position on a core manager base 267 in a removable manner.

The core manager 266 is configured to index the core manager tray 258 along a pair of guides 268, either manually or with an automated system (not shown), to a series of positions relative to the micro-coring device 250. The indexed positions can be predetermined according to the specific size and capacity of the micro-coring device 250.

For example, in FIG. 31, the core manager 266 has disposed the core manager tray 258 and carrier medium 257 in a first indexed position on the core manager base 267. Following the harvesting of a set of cores from a donor site, an operator places the micro-coring device 250 onto the core manager base 267. With the core manager tray 258 and carrier medium 257 in place, the micro-coring device 250 can be operated to remove the cores from the coring tools 223 and place them onto the carrier medium 257.

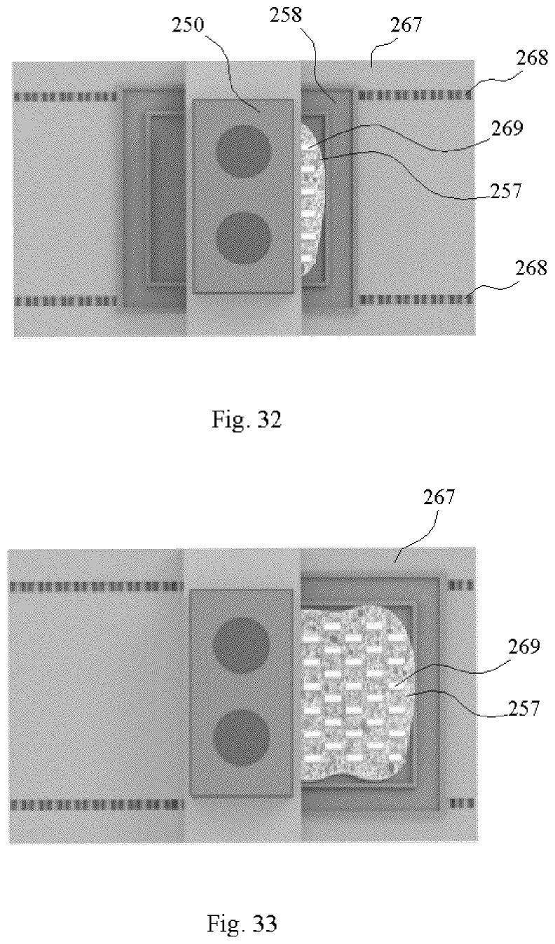

Following placement of the cores onto the carrier medium 257, the operator can remove the micro-coring device 250 from the core manager system 260 and return it to the donor site to harvest additional cores. Following the harvesting, as shown in FIG. 32, the operator can return the micro-coring device 250 to the core manager system 260 and place it back into position on the core manager base 267. The core manager tray 258 is then indexed on the guides 268 into its next position relative to the micro-coring device 250. The micro-coring device 250 extract the second set of cores and place the cores onto the carrier medium 257.

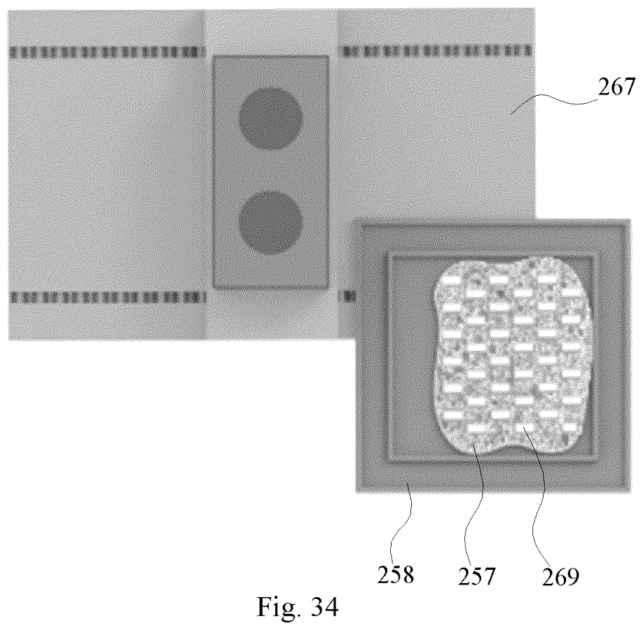

For example, a portion of the carrier medium 257 is illustrated as being populated by cores 269 when in the position illustrated in FIG. 31. In FIG. 33, the carrier medium 257 has been fully populated with cores 269 and can be removed from the core manager base 267. FIG. 34 shows the core manager tray 258 removed from the core manager base 267. The carrier medium 257 is fully populated with cores 269 and can be placed onto the wound site and dressed. The cores 269 in this arrangement have been laid flat on the carrier medium 257. In other arrangements, the cores 269 may be placed in a vertical, or other, orientation.

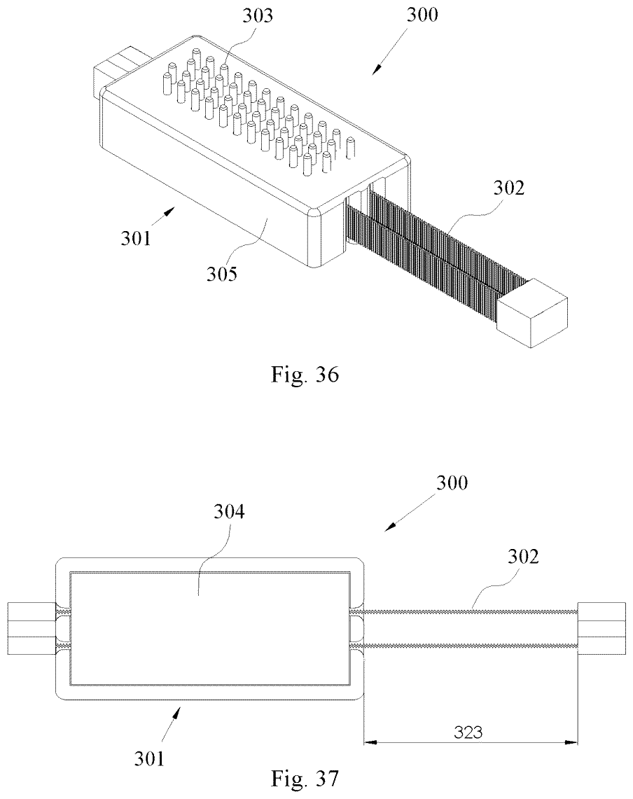

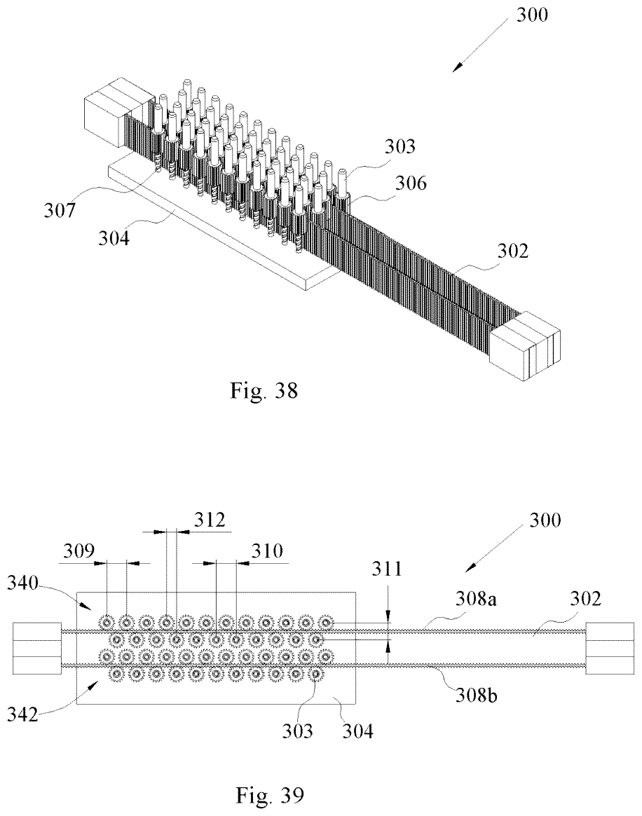

FIGS. 36 and 37 illustrate an arrangement of a micro-coring device 300 of the current innovation. As shown, the micro-coring device 300 includes a housing 301, pull strip 302 and a plurality of harvesting tools 303. As shown in FIG. 36, the harvesting tools 301 are disposed in a fully extended position. The housing 301 includes a base 304 and cover 305. FIG. 38 illustrates an isometric view of the micro-coring device 300 with the cover 305 removed. Each harvesting tool 303 includes an attached gear 306 and defines internal threads that mesh with the external threads of the extractor pins 307, which are fixed to the base 304. The pull strip 302 meshes with the gears 306 such that as the pull strip 302 is actuated, the pull strip 302 rotates the gears 306 which, in turn, rotate the harvesting tools 303. As the harvesting tools 303 rotate, their internal threads mesh with the external threads of the extractor pins 307, causing the harvesting tools 303 to extend or retract, depending upon the direction of the actuation of the pull strip 302.

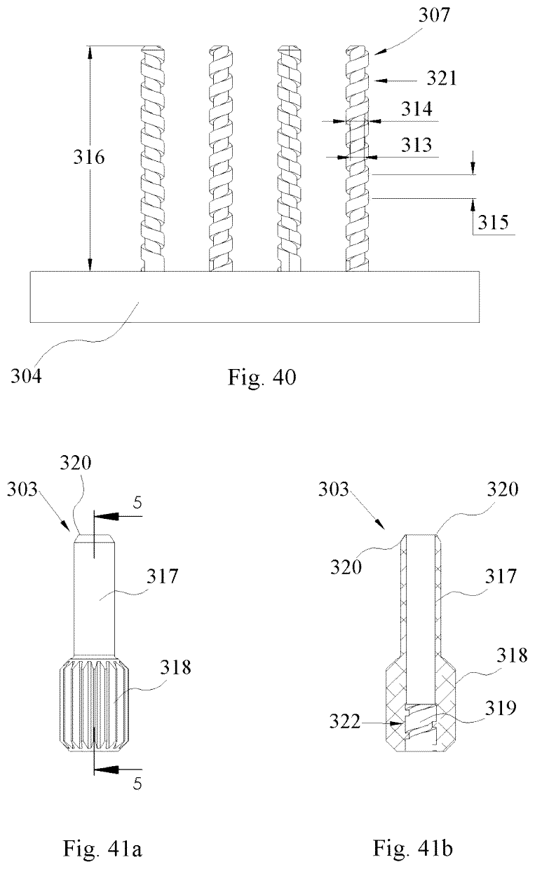

FIG. 39 is a top view of the micro-coring device 300 with the cover 305 removed. In this arrangement, forty-six harvesting tools 303 are arrayed in two pairs 340, 342 of two staggered rows each. The pull strip 302 includes two actuating segments 308a, 308b with one segment 308a, 308b bisecting each pair 340, 342 of harvesting tool 303 rows. Each pair of harvesting tools 303 includes one row of twelve harvesting tools 303 separated by a distance 309 of approximately 5 mm and one row of eleven harvesting tools 303, separated by a distance 310 of approximately 5 mm. The rows are arrayed on either side of each actuating segment 308a, 308b at a distance 311 of approximately 4.5 mm and staggered at a distance 312 of approximately 2.5 mm, or half the distance between harvesting tools 303. Actual dimensions, as well as number and arrangement of harvesting tools 303 can be determined by particular application and design choices. While this example shows two pairs of harvesting tools 303, a device with only one pair or with more than two pairs are within the scope of this innovation.

FIG. 40 illustrates an end view of an arrangement of the base 304 with extractor pins 307 attached. The outside diameter 321 of the extractor pins 307 includes an external screw thread. FIG. 41a is a side view, and FIG. 41b is a section view taken through section 5-5 of FIG. 41a of an arrangement of a harvesting tool 303. The harvesting tool 303 includes a coring section 317 with a sharpened end 320 and attached gear 318. The coring tool 303 may be made using a separate coring section and gear that are adhered together, or may be a made as a single piece, for example, 3D printed from a suitable material. The inside diameter 322 of the gear 318 includes an internal screw thread designed to mesh with the external screw thread of the extractor pin 307. The threads of the gears 318 and extractor pins 307 can include both right and left handed thread designs. The pull strip 302 actuates a row of gears 318 on either side, causing them to rotate in opposite directions. To compensate for this, the threads on one side of the pull strip 302 are configured as right handed threads while the threads on the opposite side of the pull strip are configured as left handed threads.

Any suitable thread design produces the desired engagement and pitch can be used. The following is an example of one arrangement of a thread design that utilizes a square thread design, similar in shape to an ACME thread. The minor diameter 313 is approximately 1 mm, the major diameter 314 is approximately 1.5 mm and the thread pitch 304 is approximately 1.5 mm. With this design, every revolution of the harvesting tool 303 produces a vertical movement of 1.5 mm.

The design of the gear 318 can be related to particular application and design choices. In one arrangement, the gear 318 may have a pitch diameter of approximately 3.8 mm with 18 teeth that are approximately 0.5 mm high. The pull strip actuating segments 308a, 308b can have the same tooth size and shape as the gears 318. Design of a rack and pinion type gear arrangement is known in the art and any suitable design may be used.

When the pull strip 302 is actuated, the distance that the sharpened end 320 of the coring tool 303 penetrates the tissue at the donor site is determined by the distance 323 (FIG. 37) of actuation of the pull strip 302. For example, if a penetration depth of 6 mm is desired, using the dimensions of the above example, where each rotation of the coring tool 303 advances the tool 1.5 mm, a total of 4 rotations is needed. With a pitch diameter of 3.8 mm, the pull strip 302 can travel a distance of 15.2 mm. Various methods may be used to control the insertion depth. For example, the length of the pull strip 302 can be chosen to allow a predetermined length of pull 323; the device may be assembled with the desired length of pull strip 302 extended from the housing 301 to provide the desired penetration; a stop device (not shown) may be placed onto the pull strip 302 at a location that allows partial actuation; an index or scale (not shown) may be included on the pull strip 302 to allow the user to see when the pull strip 302 has reached the desired location.

In use, the penetration depth of the coring tools 303 is preset using any of the methods described in the above examples. The micro-coring tool 300 is then placed and held onto the prepared skin at the donor site, and the pull strip 302 is actuated to extend the coring tools 303 into the donor tissue. The micro-coring device 300 is then lifted from the donor site, retaining the cores inside the coring tools 303. The micro-coring tool 300 is then moved either to a carrier medium or directly to a wound site. The pull strip 302 is then actuated in the opposite direction, causing the cores to be extracted and placed onto the carrier medium or donor site. This operation can be repeated multiple times as needed to harvest the number of cores required to form an autograft. Transfer can be accomplished using a core manager 266 as seen in FIGS. 31 through 34. Alternatively, the cores may be transferred from the micro-coring device 300 directly onto a carrier medium or wound site.

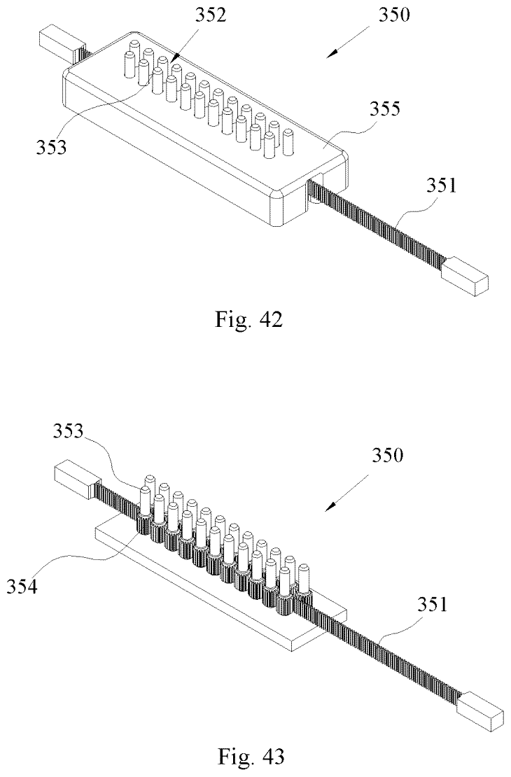

FIG. 42 illustrates an isometric view of an arrangement of a micro-coring device 350 that utilizes a single pull strip 351 and one pair 352 of staggered rows of coring tools 353. While shown as a single pull, single pair device, this arrangement may also be used with two or more pairs of coring tools as with previous arrangements. This arrangement, however, does not use a threaded extractor pin or threaded gear segment. FIG. 43 illustrates an isometric view of the coring device 350 of FIG. 42 with the top housing 355 removed. The coring tools 353 include a gear section 354 as described with previous arrangements. In this arrangement, as the pull strip 351 is actuated, it causes the gears 354 and therefore the entire coring tool 353 to rotate. Since there is no threaded extractor pin arrangement, the coring tool 353 rotates but does not extend and retract but is constrained in an extended position as shown.

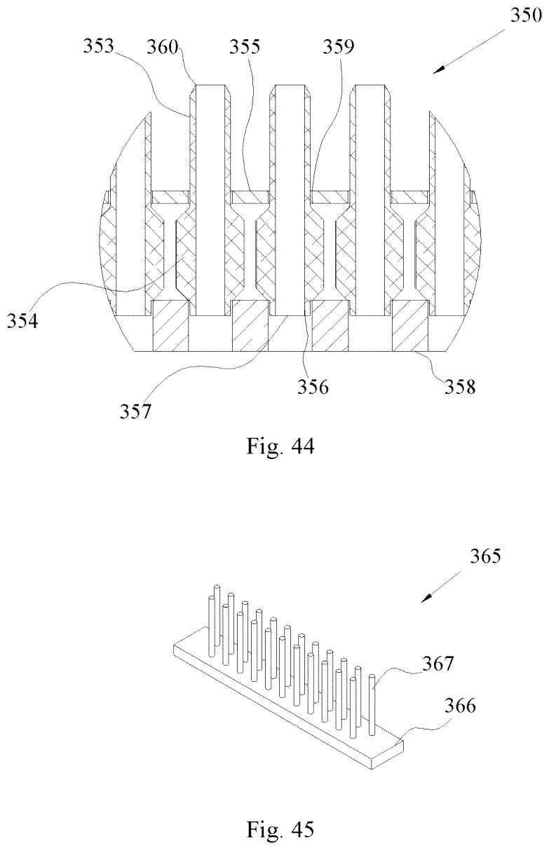

FIG. 44 illustrates an enlarged section view through one row of coring tools 353. The coring tool 353 is captured at the bottom by a boss 356 depending from the gear 354 that fits into a round recess 357 in the housing base 358 and by an opening 359 in the top housing 355 that captures the coring tool 353 above the gear 354. In this manner, the coring tools 353 are allowed to rotate but are constrained vertically as the pull strip 351 is actuated.

In use, the micro-coring device 350 is held by the user so that the sharpened ends 360 contact the tissue at the prepared donor site evenly. As the user applies pressure against the tissue, the pull strip 351 is actuated to rotate the coring tools 353. The combination of application of pressure against the tissue and rotation of the coring tools 353 causes the sharpened end 360 of the coring tools 353 to penetrate the tissue and capture cores in each coring tool 353. The micro-coring device 350 is then pulled away from the tissue, removing cores as the coring tools 353 exit the tissue.

Since the micro-coring device 350 does not include extraction pins integrally incorporated therewith, an alternative mechanism for removing the cores from the coring tools 353 is utilized. FIG. 45 illustrates an isometric view of an arrangement of an extraction device 365 having a base 366 and multiple extractor pins 367 attached thereto. The extractor pins 367 are configured to match the locations of the array of coring tools 353 of the micro-coring device 350. The length and diameter of the extractor pins 367 allows the extractor pins 367 to pass through the housing base 358 and coring tools 353 and push the harvested cored out of the coring tools 353 and onto the carrier medium or wound site. The extractor pins 367 may all be of the same length to push the harvested cores out concurrently, or alternatively may of varying lengths to push the cores out sequentially as desired.

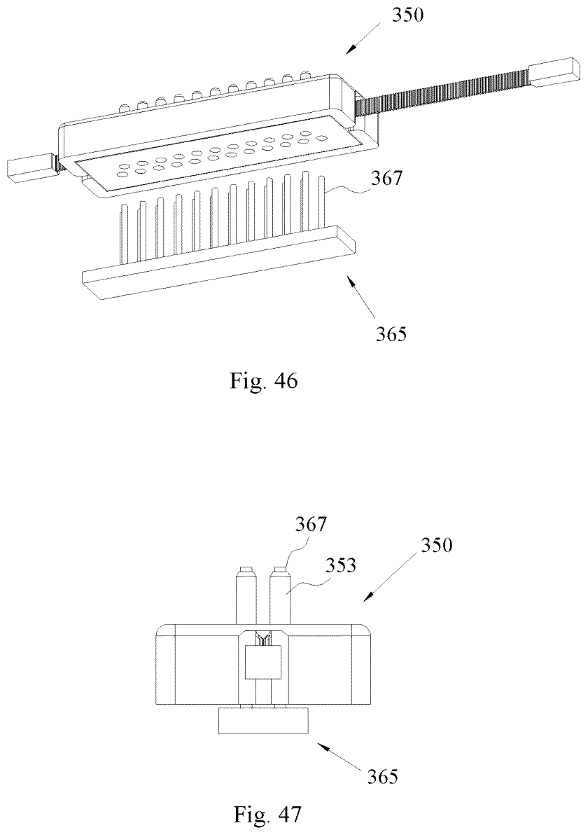

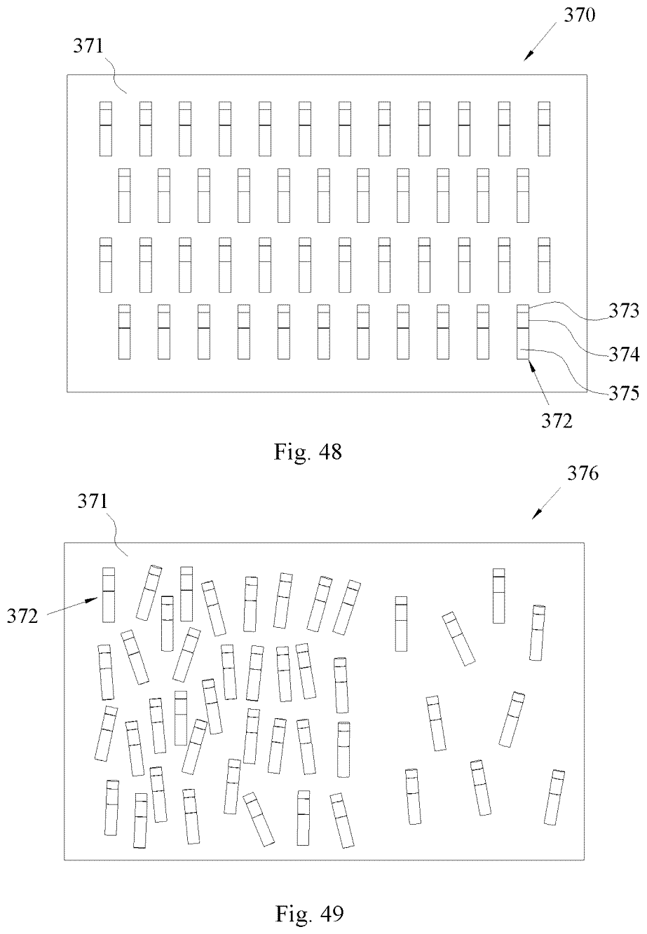

FIG. 46 illustrates an isometric view of the extraction device 365 aligned with the micro-coring device 350. The extractor pins 367 are in position to pass through the micro-coring device 350 and eject the harvested cores. FIG. 47 illustrates a side view of the micro-coring device 350 with the extraction device 365 inserted. The ends of the extractor pins 367 are illustrated as visible protruding from the coring tools 353. The harvested cores (not shown) have now been removed from the coring tools 353.

A substantially even distribution of cores at the recipient site is necessary in order to achieve a successful skin graft. The innovations shown herein provide mechanisms and methods to harvest cores in a particular pattern and density such that the cores can be transferred either directly to the recipient site or onto a carrier medium. If the cores are allowed to change position as they are transferred to the wound site, it can result in locations of higher and lower density, resulting in uneven healing.

A pattern is defined as a geometric orientation of the cores on the carrier medium. For example, the pattern can define a conventional geometric shape (i.e., triangles, squares, even or staggered rows, etc.) or a randomized grouping of cores as transferred from the micro-coring device to the carrier medium or recipient site. Density is defined as the number of cores per unit area or percentage of surface area covered by cores at the recipient site. Both pattern and density can correspond to the needs of a particular application.

FIG. 48 illustrates a top view of an example of an autograft 370. Harvested cores 372 have been placed onto the carrier medium 371. Each of the cores 372 include a layer of epidermis 373, dermis 374 and subcutis 375. The cores 372 are arrayed in a pattern such as one that would be created by one or more harvesting cycles of a micro-coring device. This figure is a visual example only and not necessarily to scale, shape, or exact layout. The cores 372 are evenly spaced and at a predetermined density. FIG. 49 illustrates a top view of an example of an autograft 376 in which the cores 372 have shifted on the carrier medium 371. The density of cores 372 on the left side of the autograft 376 is higher than that of the right side. As this graft heals, the left side provides a relatively more normal health and appearance than that of the right side.

When using a carrier medium, it is important that the harvested cores remain in their relative orientation when placed onto the wound site. This means that they retain the pattern and density in which they were placed onto the carrier medium. In order for the cores to remain in the desired orientation, the carrier medium can be configured as a wound dressing that is capable of holding the cores in place during transfer to the wound site. The wound dressing can be configured to hold the cores in place while on the wound site for at least approximately two weeks to allow the healing process to begin. Along with the ability to hold the cores in place, the wound dressing may have other attributes to aid in healing. For example:

The wound dressing can include a bacterial barrier that helps to reduce the risk of secondary infection.

The wound dressing can be configured as a relatively thick, absorbent, low-adherent pad or wound contact layer to minimize the number of dressing changes while protecting the wound from further injury. The low-adherent wound contact layer leaves the wound site clean and comfortable, reducing pain when changing the dressing.

The wound dressing can be configured with a structure having relatively high moisture vapor transmission rate (MVTR). Such a wound dressing includes a molecular structure that becomes engaged in the presence of moisture, mitigating or preventing the accumulation of moisture underneath the dressing, which reduces bacterial growth and the risk of maceration.

The wound dressing can be configured as a waterproof dressing that is impermeable or semi-impermeable to water and body fluids, thereby allowing the user to shower without changing dressings.

The wound dressing can be configured as a clear film which allows the user to monitor the level of fluid and check for infection at the wound site without removing the dressing, thus increasing wear time and limiting exposure of a healing wound.

There are a number of wound dressings that are commercially available that can be used in this application. Examples of suitable commercially available products include Telfa Non-Adherent Clear Wound Dressing manufactured by Covidien, Opsite IV 3000 Dressing and Profore WCL wound contact layer dressing, both manufactured by Smith & Nephew as is ConvaTec's AQUACEL® Ag Advantage

In one arrangement, the micro-coring device of the present innovation can be utilized in the treatment of certain scar tissues, such as hypertrophic and keloid scars. Hypertrophic scars and keloids are both raised, firm scars formed from excess fibrinogen production and collagen during healing. Each type of scar can cause severe itching, can be painful, and can cause movement restrictions and cosmetic disfigurement. Conventional treatments include compression therapy and steroids, as well as surgical treatment involving excision of the scar with primary closure. Further, radiation therapy can be used as an adjuvant treatment for keloids after surgical excision. These conventional treatments all have deficiencies such as limited effectiveness, invasive procedures, and side effects.

By contrast, in a procedure using a micro-coring device, the micro-coring device can remove cores from the damaged tissue at the scar site and replace the removed tissue with cores of healthy tissue from a donor site. In use, the micro-coring device is placed onto the tissue at the scar site and cores are removed in the same manner that cores are harvested at a donor site, as described above. The harvested scar tissue cores are ejected from the micro-coring device and discarded. The micro-coring device is then used to harvest a set of healthy cores from a donor site. The micro-coring device is returned to the same position on the scar site during removal of the scar tissue cores. The healthy cores from the donor site are now ejected from the micro-coring device directly into the openings that are left vacant in the scar site from the core removal process. The healthy cores are integrated into the scar tissue during the healing process, giving the scarred area more flexibility and normal sensation, as well as improved cosmetic appearance.

While various arrangements of the innovation have been particularly shown and described, it will be understood by those skilled in the art that various changes in form and details may be made therein without departing from the spirit and scope of the innovation as defined by the appended claims.

What is claimed is:

1. A micro-coring system, comprising:
a harvesting tool configured to harvest and transfer at least one tissue core from a donor site to a recipient site, the at least one tissue core having a diameter of between about 1.0 mm and 3.0 mm.

2. The micro-coring system of claim 1, wherein the harvesting tool is configured to transfer the harvested cores to a carrier medium.

3. The micro-coring system of claim 2, wherein the harvesting tool is configured to extract tissue cores and populate the carrier medium to generate an autograft of a predetermined size and shape.

4. The micro-coring system of claim 1, wherein the harvesting tool is configured to transfer the harvested cores to a patient wound site.

5. The micro-coring system of claim 1, wherein the harvesting tool comprises:
a coring tool configured to penetrate tissue at the donor site and capture a tissue core from the tissue, and
an extractor pin disposed within an aperture extending along a longitudinal axis of the coring tool, the extractor pin configured to extract the core from the coring tool.

6. The micro-coring system of claim 5, wherein:
the extractor pin defines an external screw thread disposed on an external surface of the extractor pin;
the coring tool defines an internal screw thread disposed on the inner surface of the coring tool and a sharpened edge disposed at a distal end;
the externally threaded portion of the extractor pin configured to mesh with the internally threaded portion of the coring tool, the coring tool configured to translate along a longitudinal axis of the extractor pin such that rotation of the coring tool in a first direction relative to the extractor pin extends the coring tool into the donor site tissue to capture a tissue core, and rotation of the coring tool in a second direction relative to the extractor pin extracts the tissue core from the coring tool.

7. The micro-coring system of claim 6, wherein the coring tool comprises a gear disposed on an outer surface of the coring tool, the gear configured rotate the coring tool (i) in a first direction relative to the extractor pin to extend the coring tool into the donor site and (ii) in a second direction relative to the extractor pin to extract the tissue core from the coring tool.

8. The micro-coring system of claim 7, wherein the gear of the coring tool meshes with a gear of at least one other coring tool such that rotation of the gear of the coring tool causes rotation of the gear of the at least one other coring tool.

9. The micro-coring system of claim 8, wherein at least one of the gear of the coring tool and the gear of at least one other coring tool is configured to be driven by one or more electric motors.

10. The micro-coring system of claim 8, wherein at least one of the gear of the coring tool and the gear of at least one other coring tool is configured to be driven by manually actuated pull strip.

11. The micro-coring system of claim 5, wherein the extractor pin comprises a longitudinal axis and defines a central aperture that extends along the longitudinal axis and configured to be disposed in fluid communication with a vacuum source.

12. The micro-coring system of claim 1, wherein the at least one tissue core comprises skin adnexa having at least one hair follicle, at least one sweat gland, and at least one sebaceous gland.

13. The micro-coring system of claim 1, wherein when harvesting and transferring the at least one tissue core from the donor site to the recipient site, the harvesting tool is configured to harvest and transfer a set of tissue cores defining between about a 10% to 20% core density from the donor site to the recipient site.

14. A method of harvesting a tissue cores from a donor site, comprising:
disposing a harvesting tool at a donor site, the harvesting tool comprising a coring tool defining an aperture extending along a longitudinal axis of the coring tool and an extractor pin disposed within the aperture defined by the coring tool;
translating the coring tool in a first direction relative to the extractor pin to penetrate tissue of the donor site;
excising a tissue core from the tissue of the donor site, the tissue core having a diameter of between about 1.0 mm and 3.0 mm diameter; and
translating the coring tool in a second direction relative to the extractor pin to extract the tissue core from the coring tool.

15. The method of claim 14 wherein:
translating the coring tool in the first direction relative to the extractor pin and the donor site to penetrate tissue of the donor site comprises rotating the coring tool in a first direction relative to the extractor pin to penetrate tissue of the donor site; and
translating the coring tool in the second direction relative to the extractor pin and the donor site to extract the tissue core from the donor site comprises rotating the coring tool in a second direction relative to the extractor pin to dispose the tissue core within the harvesting tool.

16. The method of claim 14 wherein:
translating the coring tool in the first direction relative to the extractor pin and the donor site to penetrate tissue of the donor site comprises rotating an internal screw thread disposed on an inner surface of the coring tool in a first direction relative to an external screw thread disposed on an external surface of the extractor pin; and
translating the coring tool in the second direction relative to the extractor pin and the donor site to extract the tissue core from the donor site comprises rotating the internal screw thread disposed on the inner surface of the coring tool in a second direction relative to the external screw thread disposed on an external surface of the extractor pin.

17. The method of claim 15, further comprising:
disposing the harvesting tool at a recipient site; and
continuing to translate the coring tool in a second direction relative to the extractor pin to extract the tissue core onto the recipient site.

18. The method of claim 15, further comprising:
disposing the harvesting tool at a carrier medium; and
continuing to translate the coring tool in a second direction relative to the extractor pin to extract the tissue core onto the carrier medium.

19. The method of claim 15, wherein the tissue core comprises skin adnexa having at least one hair follicle, at least one sweat gland, and at least one sebaceous gland.
